(19) United States
(12) Patent Application Publication
(10) Pub. No.: US 2025/0340645 A1
(43) Pub. Date: Nov. 6, 2025

(54) METHODS OF TREATING TUMOR

(71) Applicant: Bristol-Myers Squibb Company, Princeton, NJ (US)

(72) Inventors: Ming LEI, Pennington, NJ (US); Nathan O. SIEMERS, Pacific Grove, CA (US); Dimple PANDYA, Princeton, NJ (US); Han CHANG, West Windsor, NJ (US); Teresa K. SANCHEZ, Princeton, NJ (US); Christopher T. HARBISON, Hamilton, NJ (US); Peter M. SZABO, Pennington, NJ (US); Zachary S. BOYD, Skillman, NJ (US); Alice M. WALSH, Glenside, PA (US)

(73) Assignee: Bristol-Myers Squibb Company, Princeton, NJ (US)

(21) Appl. No.: 19/199,098

(22) Filed: May 5, 2025

Related U.S. Application Data
(63) Continuation of application No. 17/599,432, filed on Sep. 28, 2021.
(60) Provisional application No. 62/825,531, filed on Mar. 28, 2019.

(51) Int. Cl. C07K 16/28; A61K 39/00; A61P 35/00; C12Q 1/6886

(52) U.S. Cl. CPC C07K 16/2818; A61P 35/00; C07K 16/2827; C12Q 1/6886; A61K 2039/505; C07K 2317/21; C07K 2317/76; C12Q 2600/106; C12Q 2600/158

(57) ABSTRACT

The disclosure provides a method for treating a subject afflicted with a tumor comprising administering to the subject a therapeutically effective amount of an anti-PD-1 antibody or antigen-binding portion thereof or an anti-PD-L1 antibody or anti-gen-binding portion thereof, wherein the subject is identified as having a high inflammatory gene signature score. In some embodiments, the high inflammatory gene signature score is determined by measuring the expression of a panel of inflammatory genes in a tumor sample obtained from the subject, wherein the inflammatory gene panel comprises CD274 (PD-LI), CD8A, LAG3, and STAT1.

### CROSS REFERENCE TO RELATED APPLICATIONS

This PCT application claims the priority benefit of U.S. Provisional Application No. 62/825,531, filed Mar. 28, 2019, which is incorporated herein by reference in its entirety.

### FIELD OF THE DISCLOSURE

The present disclosure provides a method for treating a subject afflicted with a tumor using an immunotherapy.

### BACKGROUND OF THE DISCLOSURE

Human cancers harbor numerous genetic and epigenetic alterations, generating neoantigens potentially recognizable by the immune system (Sjoblom et al., Science (2006) 314(5797):268-274). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

Until recently, cancer immunotherapy had focused substantial effort on approaches that enhance anti-tumor immune responses by adoptive-transfer of activated effector cells, immunization against relevant antigens, or providing non-specific immune-stimulatory agents such as cytokines. In the past decade, however, intensive efforts to develop specific immune checkpoint pathway inhibitors have begun to provide new immunotherapeutic approaches for treating cancer, including the development of antibodies such as nivolumab and pembrolizumab (formerly lambrolizumab; USAN Council Statement, 2013) that bind specifically to the Programmed Death-1 (PD-1) receptor and block the inhibitory PD-1/PD-1 ligand pathway (Topalian et al., 2012a, b; Topalian et al., 2014; Hamid et al., 2013; Hamid and Carvajal, 2013; McDermott and Atkins, 2013).

PD-1 is a key immune checkpoint receptor expressed by activated T and B cells and mediates immunosuppression. PD-1 is a member of the CD28 family of receptors, which includes CD28, CTLA-4, ICOS, PD-1, and BTLA. Two cell surface glycoprotein ligands for PD-1 have been identified, Programmed Death Ligand-1 (PD-L1) and Programmed Death Ligand-2 (PD-L2), that are expressed on antigen-presenting cells as well as many human cancers and have been shown to downregulate T cell activation and cytokine secretion upon binding to PD-1. Inhibition of the PD-1/PD-L1 interaction mediates potent antitumor activity in preclinical models (U.S. Pat. Nos. 8,008,449 and 7,943,743), and the use of antibody inhibitors of the PD-1/PD-L1 interaction for treating cancer has entered clinical trials (Brahmer et al., 2010; Topalian et al., 2012a; Topalian et al., 2014; Hamid et al., 2013; Brahmer et al., 2012; Flies et al., 2011; Pardoll, 2012; Hamid and Carvajal, 2013).

Nivolumab (formerly designated 5C4, BMS-936558, MDX-1106, or ONO-4538) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014). Nivolumab has shown activity in a variety of advanced solid tumors, including renal cell carcinoma (renal adenocarcinoma, or hypernephroma), melanoma, and non-small cell lung cancer (NSCLC) (Topalian et al., 2012a; Topalian et al., 2014; Drake et al., 2013; WO 2013/173223).

The immune system and response to immuno-therapy are complex. Additionally, anti-cancer agents can vary in their effectiveness based on the unique patient characteristics. Accordingly, there is a need for targeted therapeutic strategies that identify patients who are more likely to respond to a particular anti-cancer agent and, thus, improve the clinical outcome for patients diagnosed with cancer.

### SUMMARY OF THE DISCLOSURE

Certain aspects of the present disclosure are directed to a method for treating a human subject afflicted with a tumor comprising (i) identifying a subject exhibiting a high inflammatory signature score; and (ii) administering to the subject an anti-PD-1 antibody; wherein the inflammatory signature score is determined by measuring the expression of a panel of inflammatory genes (“inflammatory gene panel”) in a tumor sample obtained from the subject; and wherein the inflammatory gene panel comprises CD274 (PD-L1), CD8A, LAG3, and STAT1.

Certain aspects of the present disclosure are directed to a method for treating a human subject afflicted with a tumor comprising administering an anti-PD-1 antibody to the subject, wherein the subject is identified as exhibiting a high inflammatory signature score prior to the administration; wherein the inflammatory signature score is determined by measuring the expression of a panel of inflammatory genes (“inflammatory gene panel”) in a tumor sample obtained from the subject; and wherein the inflammatory gene panel comprises CD274 (PD-L1), CD8A, LAG3, and STAT.

Certain aspects of the present disclosure are directed to a method for identifying a human subject afflicted with a tumor suitable for an anti-PD-1 antibody treatment comprising (i) measuring an inflammatory signature score in a tumor sample obtained from the subject and (ii) administering to the subject an anti-PD-1 antibody if the subject exhibits a high inflammatory signature score; wherein the inflammatory signature score is determined by measuring the expression of a panel of inflammatory genes (“inflammatory gene panel”) in the tumor sample obtained from the subject; and wherein the inflammatory gene panel comprises CD274 (PD-L1), CD8A, LAG3, and STAT.

In some embodiments, the inflammatory gene panel consists of less than about 20, less than about 18, less than about 15, less than about 13, less than about 10, less than about 9, less than about 8, less than about 7, less than about 6, or less than about 5 inflammatory genes. In some embodiments, the inflammatory gene panel consists essentially of (i) CD274 (PD-L1), CD8A, LAG3, and STAT, and (ii) 1 additional inflammatory gene, 2 additional inflammatory genes, 3 additional inflammatory genes, 4 additional inflammatory genes, 5 additional inflammatory genes, 6 additional inflammatory genes, 7 additional inflammatory genes, 8 additional inflammatory genes, 9 additional inflammatory genes, 10 additional inflammatory genes, 11 additional inflammatory genes, 12 additional inflammatory genes, 13 additional inflammatory genes, 14 additional inflammatory genes, or 15 additional inflammatory genes.

In some embodiments, the additional inflammatory gene is selected from the group consisting of CCL2, CCL3, CCL4, CCL5, CCR5, CD27, CD274, CD276, CMKLR1, CXCL10, CXCL11, CXCL9, CXCR6, GZMA, GZMK, HLA-DMA, HLA-DMB, HLA-DOA, HLA-DOB, HLA-DQA1, HLA-DRA, HLA-DRB1, HLA-E, ICOS, IDO1, IFNG, IRF1, NKG7, PDCD ILG2, PRF1, PSMB10, TIGIT, and any combination thereof.

In some embodiments, the inflammatory gene panel consists essentially of CD274 (PD-L1), CD8A, LAG3, and STAT1. In some embodiments, the inflammatory gene panel consists of CD274 (PD-L1), CD8A, LAG3, and STAT.

In some embodiments, the high inflammatory signature score is characterized by an inflammatory signature score that is greater than an average inflammatory signature score, wherein the average inflammatory signature score is determined by averaging the expression of the panel of inflammatory genes in tumor samples obtained from a population of subjects afflicted with the tumor.

In some embodiments, the average inflammatory signature score is determined by averaging the expression of the panel of inflammatory genes in tumor samples obtained from the population of subjects.

In some embodiments, the high inflammatory signature score is characterized by an inflammatory signature score that is at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 125%, at least about 150%, at least about 175%, at least about 200%, at least about 225%, at least about 250%, at least about 275%, or at least about 300% higher than the average inflammatory signature score. In some embodiments, the high inflammatory signature score is characterized by an inflammatory signature score that is at least about 50% higher than the average inflammatory signature score. In some embodiments, the high inflammatory signature score is characterized by an inflammatory signature score that is at least about 75% higher than the average inflammatory signature score.

In some embodiments, the tumor sample is a tumor tissue biopsy. In some embodiments, the tumor sample is a formalin-fixed, paraffin-embedded tumor tissue or a fresh-frozen tumor tissue. In some embodiments, the expression of the inflammatory genes in the inflammatory gene panel is determined by detecting the presence of inflammatory gene mRNA, the presence of a protein encoded by the inflammatory gene, or both. In some embodiments, the presence of inflammatory gene mRNA is determined using reverse transcriptase PCR. In some embodiments, the presence of the protein encoded by the inflammatory gene is determined using an IHC assay. In some embodiments, the IHC assay is an automated IHC assay.

In some embodiments, the anti-PD-1 antibody cross-competes with nivolumab for binding to human PD-1. In some embodiments, the anti-PD-1 antibody binds to the same epitope as nivolumab. In some embodiments, the anti-PD-1 antibody is a chimeric, humanized or human monoclonal antibody or a portion thereof. In some embodiments, the anti-PD-1 antibody comprises a heavy chain constant region which is of a human IgG1 or IgG4 isotype. In some embodiments, the anti-PD-1 antibody is nivolumab. In some embodiments, the anti-PD-1 antibody is pembrolizumab.

In some embodiments, the anti-PD-1 antibody is administered at a dose ranging from at least about 0.1 mg/kg to at least about 10.0 mg/kg body weight once about every 1, 2 or 3 weeks. In some embodiments, the anti-PD-1 antibody is administered at a dose of at least about 3 mg/kg body weight once about every 2 weeks. In some embodiments, the anti-PD-1 antibody or antigen-binding portion thereof is administered at a flat dose. In some embodiments, the anti-PD-1 antibody or antigen-binding portion thereof is administered at a flat dose of at least about 200, at least about 220, at least about 240, at least about 260, at least about 280, at least about 300, at least about 320, at least about 340, at least about 360, at least about 380, at least about 400, at least about 420, at least about 440, at least about 460, at least about 480, at least about 500 or at least about 550 mg. In some embodiments, the anti-PD-1 antibody or antigen-binding portion thereof is administered at a flat dose of about 240 mg. In some embodiments, the anti-PD-1 antibody or antigen-binding portion thereof is administered at a flat dose of about 480 mg.

In some embodiments, the anti-PD-1 antibody or antigen-binding portion thereof is administered at a flat dose about once every 1, 2, 3 or 4 weeks. In some embodiments, the anti-PD-1 antibody or antigen-binding portion thereof is administered at a flat dose or about 240 mg once about every two weeks. In some embodiments, the anti-PD-1 antibody or antigen-binding portion thereof is administered at a flat dose of about 480 mg once about every four weeks.

In some embodiments, the anti-PD-1 antibody is administered for as long as clinical benefit is observed or until unmanageable toxicity or disease progression occurs. In some embodiments, the anti-PD-1 antibody is formulated for intravenous administration. In some embodiments, the anti-PD-1 antibody is administered at a subtherapeutic dose.

In some embodiments, the method further comprises administering an antibody or an antigen binding fragment thereof that binds specifically to cytotoxic T-lymphocyte-associated protein 4 (CTLA-4) (“an anti-CTLA-4 antibody”). In some embodiments, the anti-CTLA-4 antibody cross-competes with ipilimumab or tremelimumab for binding to human CTLA-4. In some embodiments, the anti-CTLA-4 antibody binds to the same epitope as ipilimumab or tremelimumab. In some embodiments, the anti-CTLA-4 antibody is ipilimumab. In some embodiments, the anti-CTLA-4 antibody is tremelimumab.

In some embodiments, the anti-CTLA-4 antibody is administered at a dose ranging from 0.1 mg/kg to 20.0 mg/kg body weight once every 2, 3, 4, 5, 6, 7, or 8 weeks. In some embodiments, the anti-CTLA-4 antibody is administered at a dose of 1 mg/kg body weight once every 6 weeks. In some embodiments, the anti-CTLA-4 antibody is administered at a dose of 1 mg/kg body weight once every 4 weeks.

In some embodiments, the anti-CTLA-4 antibody is administered at a flat dose. In some embodiments, the anti-CTLA-4 antibody is administered at a flat dose of at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, or at least about 200 mg. In some embodiments, the anti-CLTA-4 antibody is administered as a flat dose about once every 2, 3, 4, 5, 6, 7, or 8 weeks.

In some embodiments, the tumor is derived from a cancer selected from the group consisting of hepatocellular cancer, gastroesophageal cancer, melanoma, bladder cancer, lung cancer, kidney cancer, head and neck cancer, colon cancer, and any combination thereof. In some embodiments, the tumor is derived from a hepatocellular cancer. In some embodiments, the tumor is derived from a gastroesophageal cancer. In some embodiments, the tumor is derived from a melanoma.

In some embodiments, the tumor is relapsed. In some embodiments, the tumor is refractory. In some embodiments, the tumor is refractory following at least one prior therapy comprising administration of at least one anticancer agent. In some embodiments, the at least one anticancer agent comprises a standard of care therapy. In some embodiments, the at least one anticancer agent comprises an immunotherapy.

In some embodiments, the tumor is locally advanced. In some embodiments, the tumor is metastatic.

In some embodiments, the administering treats the tumor. In some embodiments, the administering reduces the size of the tumor. In some embodiments, the size of the tumor is reduced by at least about 10%, about 20%, about 30%, about 40%, or about 50% compared to the tumor size prior to the administration. In some embodiments, the subject exhibits progression-free survival of at least about one month, at least about 2 months, at least about 3 months, at least about 4 months, at least about 5 months, at least about 6 months, at least about 7 months, at least about 8 months, at least about 9 months, at least about 10 months, at least about 11 months, at least about one year, at least about eighteen months, at least about two years, at least about three years, at least about four years, or at least about five years after the initial administration.

In some embodiments, the subject exhibits stable disease after the administration. In some embodiments, the subject exhibits a partial response after the administration. In some embodiments, the subject exhibits a complete response after the administration.

Certain aspects of the present disclosure are directed to a kit for treating a subject afflicted with a tumor, the kit comprising: (a) a dosage ranging from about 4 mg to about 500 mg of an anti-PD-1 antibody; and (b) instructions for using the anti-PD-1 antibody in any method disclosed herein. In some embodiments, the kit further comprises an anti-CTLA-4 antibody. In some embodiments, the kit further comprises an anti-PD-L1 antibody.

### BRIEF DESCRIPTION OF THE DRAWINGS

FIG. 1 is a schematic representation of the study design for exploratory endpoint biomarker assessments of efficacy of NIVO in patients with advanced hepatocellular carcinoma (HCC) and with (“SOR-experienced”) and without (“SOR-naïve”) prior sorafenib (SOR) treatment in the clinical trial NCT01658878.

FIGS. 2A and 2B are waterfall plots illustrating the best reduction from baseline in target lesions (%) in all subjects in the overall populations (FIG. 2A) and the SOR-experienced population (FIG. 2B), wherein subjects in each plot are labeled according to PD-L1 status. FIGS. 2C and 2D are graphical representations of the overall survival (months) of patients in the overall population (SOR-naïve and SOR-experienced; FIG. 2C) and the SOR-experienced population alone (FIG. 2D) in patients with tumor cell PD-L1≥1% or <1%, as indicated. The number of patients at risk for each PD-L1 group is indicated below the x-axis.

FIGS. 3A-3D are plots showing the relationship between the best overall response and the percent of cells expressing a T-cell marker selected from CD3 (FIG. 3A), CD4 (FIG. 3B), CD8 (FIG. 3C), and FOXP3 (FIG. 3D) for the overall population (SOR-naïve and SOR-experienced).

FIGS. 4A-4D are graphical representations illustrating the overall survival for the overall population (SOR-naïve and SOR-experienced) stratified into tertiles based on expression of the lowest, the middle, or the highest levels of a T-cell marker selected from CD3 (FIG. 4A), CD4 (FIG. 4B), CD8 (FIG. 4C), and FOXP3 (FIG. 4D). The number of patients at risk for each stratification group is indicated below the x-axis.

FIG. 5A-5B are plots showing the relationship between the best overall response and the percent of cells expressing a macrophage marker selected from CD68 (FIG. 5A) and CD163 (FIG. 5B) for the overall population (SOR-naïve and SOR-experienced).

FIGS. 6A-6B are graphical representations illustrating the overall survival for the overall population (SOR-naïve and SOR-experienced) stratified into tertiles based on expression of the lowest, the middle, or the highest levels of a T-cell marker selected from CD68 (FIG. 6A) and CD163 (FIG. 6B). The number of patients at risk for each stratification group is indicated below the x-axis.

FIG. 7A is a plot showing the relationship between the best overall response and 4-gene signature score, described herein. FIG. 7B is a graphical representation illustrating the overall survival for the overall population (SOR-naïve and SOR-experienced) stratified into tertiles based on expression of the lowest, the middle, or the highest 4-gene inflammatory signature scores. The number of patients at risk for each stratification group is indicated below the x-axis.

FIG. 8 is a schematic showing the study design for exploratory endpoint biomarker assessments of the efficacy of nivolumab treatment with and without ipilimumab in patients with chemotherapy-refractory gastroesophageal cancer in the phase I/II clinical trial NCT01928394.

FIGS. 9A-9B are plots showing the relationship between the best overall response and tumor PD-L1 expression (FIG. 9A) and PD-L1 combined positive score (CPS; FIG. 9B), as defined herein, for subjects treated with nivolumab 3 mg/kg monotherapy or nivolumab 1 mg/kg+ipilimumab 3 mg/kg, nivolumab 3 mg/kg+ipilimumab 1 mg/kg, or nivolumab 1 mg/kg+ipilimumab 1 mg/kg.

FIGS. 10A-10F are graphical representations of the overall survival of patients in all treatment arms, stratified by tumor PD-L1 expression of ≥1% or <1% (FIG. 10A), ≥5% or <5% (FIG. 10B), ≥10% or <10% (FIG. 10C), or stratified by PD-L1 CPS of ≥1 or <1 (FIG. 10D), ≥5 or <5 (FIG. 10E), ≥10 or <10 (FIG. 10F) as indicated. The number of patients at risk for each PD-L1 group is indicated below the x-axis.

FIGS. 11A-11D are graphical representations of the overall survival of patients in the nivolumab 1 mg/kg+ipilimumab 3 mg/kg treatment arm, stratified by tumor PD-L1 expression of ≥1% or <1% (FIG. 11A) or stratified by PD-L1 CPS of ≥1 or <1 (FIG. 11B), ≥5 or <5 (FIG. 11C), ≥10 or <10 (FIG. 11D) as indicated. The number of patients at risk for each PD-L1 group is indicated below the x-axis.

FIGS. 12A-12D are plots showing the relationship between the best overall response and CD8 T-cell signature (FIG. 12A), PD-L1 transcript (FIG. 12B), Ribas 10-gene signature (FIG. 12C), and the 4-gene inflammatory signature described herein (FIG. 12D).

FIG. 13 shows the ROC analysis of the 4-gene immune signature and benefit.

FIG. 14 is a schematic of the study design for exploratory endpoints biomarker assessments of the efficacy of nivolumab monotherapy, ipilimumab monotherapy, and nivolumab/ipilimumab combination therapy in patients with unresectable stage III or IV melanoma in the NCT01721772 and the NCT01844505 trials.

FIGS. 15A-15B are Kaplan-Meier plots of the primary findings, progression free survival (PFS; FIG. 15A) and overall survival (OS; FIG. 15B) for the intent-to-treat (ITT) populations from NCT01844505 (FIGS. 15A-15B).

FIG. 16 is a bar graph showing the sample disposition of subjects treated with nivolumab+ipilimumab combination therapy, nivolumab monotherapy, or ipilimumab monotherapy in NCT01844505 and evaluated for 4-gene signature score. The total number for each group is indicated above each bar.

FIG. 17 is a plot showing the relationship between the best overall response and the 4-gene inflammatory signature score described herein in subjects administered a nivolumab/ipilimumab combination therapy, a nivolumab monotherapy, or an ipilimumab monotherapy in the NCT01844505 trial.

FIGS. 18A-18C are graphical representations illustrating the progression-free survival of subjects administered a nivolumab/ipilimumab combination therapy (FIG. 18A), a nivolumab monotherapy (FIG. 18B), or an ipilimumab monotherapy (FIG. 18C), wherein the subjects are stratified according to high 4-gene inflammatory signature score (“High ISS”) or low 4-gene inflammatory signature score (“Low ISS”). The number of patients at risk for each stratification group is indicated below the x-axis. FIG. 18D shows the corresponding hazard ratios.

FIGS. 19A-19C are graphical representations illustrating the overall survival (OS) of subjects administered a nivolumab/ipilimumab combination therapy (FIG. 19A), a nivolumab monotherapy (FIG. 19B), or an ipilimumab monotherapy (FIG. 19C), wherein the subjects are based having a high 4-gene inflammatory signature score (“High ISS”) or a low 4-gene inflammatory signature score (“Low ISS”). The number of patients at risk for each stratification group is indicated below the x-axis. FIG. 19D shows the corresponding hazard ratios.

### DETAILED DESCRIPTION OF THE DISCLOSURE

The present disclosure provides a method for treating a human subject afflicted with a tumor comprising (i) identifying a subject displaying a high inflammatory signature score; and (ii) administering to the subject a PD-1 inhibitor, e.g., an anti-PD-1 antibody or an anti-PD-L1 antibody. The present disclosure also provides a method for treating a human subject afflicted with a tumor comprising administering a PD-1 inhibitor, e.g., an anti-PD-1 antibody or an anti-PD-L1 antibody, wherein the subject is identified as having a high inflammatory signature score prior to the administration. The present disclosure also provides a method for identifying a human subject afflicted with a tumor suitable for a PD-1 inhibitor, e.g., an anti-PD-1 antibody or an anti-PD-L1 antibody, treatment comprising (i) measuring an inflammatory signature score in a tumor sample obtained from the subject and (ii) administering to the subject a PD-1 inhibitor, e.g., an anti-PD-1 antibody or an anti-PD-L1 antibody if the subject has a high inflammatory signature score.

### I. Terms

In order that the present disclosure can be more readily understood, certain terms are first defined. As used in this application, except as otherwise expressly provided herein, each of the following terms shall have the meaning set forth below. Additional definitions are set forth throughout the application.

“Administering” refers to the physical introduction of a composition comprising a therapeutic agent to a subject, using any of the various methods and delivery systems known to those skilled in the art. Preferred routes of administration for the immunotherapy, e.g., the anti-PD-1 antibody or the anti-PD-L1 antibody, include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Other non-parenteral routes include an oral, topical, epidermal or mucosal route of administration, for example, intranasally, vaginally, rectally, sublingually or topically. Administering can also be performed, for example, once, a plurality of times, and/or over one or more extended periods.

An “adverse event” (AE) as used herein is any unfavorable and generally unintended or undesirable sign (including an abnormal laboratory finding), symptom, or disease associated with the use of a medical treatment. For example, an adverse event can be associated with activation of the immune system or expansion of immune system cells (e.g., T cells) in response to a treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region is comprises one constant domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. Therefore, the term “anti-PD-1 antibody” includes a full antibody having two heavy chains and two light chains that specifically binds to PD-1 and antigen-binding portions of the full antibody. Non limiting examples of the antigen-binding portions are shown elsewhere herein.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

An “isolated antibody” refers to an antibody that is substantially free of other antibodies having different antigenic specificities (e.g., an isolated antibody that binds specifically to PD-1 is substantially free of antibodies that bind specifically to antigens other than PD-1). An isolated antibody that binds specifically to PD-1 may, however, have cross-reactivity to other antigens, such as PD-1 molecules from different species. Moreover, an isolated antibody can be substantially free of other cellular material and/or chemicals.

The term “monoclonal antibody” (mAb) refers to a non-naturally occurring preparation of antibody molecules of single molecular composition, i.e., antibody molecules whose primary sequences are essentially identical, and which exhibits a single binding specificity and affinity for a particular epitope. A monoclonal antibody is an example of an isolated antibody. Monoclonal antibodies can be produced by hybridoma, recombinant, transgenic or other techniques known to those skilled in the art.

A “human antibody” (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibody” and “fully human antibody” and are used synonymously.

A “humanized antibody” refers to an antibody in which some, most or all of the amino acids outside the CDRs of a non-human antibody are replaced with corresponding amino acids derived from human immunoglobulins. In one embodiment of a humanized form of an antibody, some, most or all of the amino acids outside the CDRs have been replaced with amino acids from human immunoglobulins, whereas some, most or all amino acids within one or more CDRs are unchanged. Small additions, deletions, insertions, substitutions or modifications of amino acids are permissible as long as they do not abrogate the ability of the antibody to bind to a particular antigen. A “humanized antibody” retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen antibody” refers to an antibody that binds specifically to the antigen. For example, an anti-PD-1 antibody binds specifically to PD-1, an anti-PD-L1 antibody binds specifically to PD-L1, and an anti-CTLA-4 antibody binds specifically to CTLA-4.

An “antigen-binding portion” of an antibody (also called an “antigen-binding fragment”) refers to one or more fragments of an antibody that retain the ability to bind specifically to the antigen bound by the whole antibody. It has been shown that the antigen-binding function of an antibody can be performed by fragments of a full-length antibody. Examples of binding fragments encompassed within the term “antigen-binding portion” of an antibody, e.g., an anti-PD-1 antibody or an anti-PD-L1 antibody described herein, include (i) a Fab fragment (fragment from papain cleavage) or a similar monovalent fragment consisting of the V_{L}, V_{H}, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

A “cancer” refers a broad group of various diseases characterized by the uncontrolled growth of abnormal cells in the body. Unregulated cell division and growth divide and grow results in the formation of malignant tumors that invade neighboring tissues and can also metastasize to distant parts of the body through the lymphatic system or bloodstream.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

“Programmed Death-1” (PD-1) refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863.

“Programmed Death Ligand-1” (PD-L1) is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-LT, and analogs having at least one common epitope with hPD-L1. The complete hPD-L1 sequence can be found under GenBank Accession No. Q9NZQ7. The human PD-L1 protein is encoded by the human CD274 gene (NCBI Gene ID: 29126).

As used herein, a PD-1 or PD-L1 “inhibitor,” refers to any molecule capable of blocking, reducing, or otherwise limiting the interaction between PD-1 and PD-L1 and/or the activity of PD-1 and/or PD-L1. In some aspects, the inhibitor is an antibody or an antigen-binding fragment of an antibody. In other aspects, the inhibitor comprises a small molecule.

“T-Cell surface glycoprotein CD8 alpha chain” or “CD8A” as used herein refers to an integral membrane glycoprotein that is involved in the immune response and that serves multiple functions in responses against both external and internal offenses. In T-cells, CD8a functions primarily as a co-receptor for MHC class I molecule/peptide complex. CD8A interacts simultaneously with the T-cell receptor (TCR) and the MHC class I proteins presented by antigen presenting cells (APCs). In turn, CD8a recruits the Src kinase LCK to the vicinity of the TCR-CD3 complex. LCK then initiates different intracellular signaling pathways by phosphorylating various substrates ultimately leading to lymphokine production, motility, adhesion and activation of cytotoxic T-lymphocytes (CTLs). This mechanism enables CTLs to recognize and eliminate infected cells and tumor cells. In NK-cells, the presence of CD8A homodimers at the cell surface provides a survival mechanism allowing conjugation and lysis of multiple target cells. CD8A homodimer molecules also promote the survival and differentiation of activated lymphocytes into memory CD8 T-cells. The complete CD8a amino acid sequence can be found under UniProtKB identification number P01732. The human CD8a protein is encoded by the human CD8a gene (NCBI Gene ID: 925).

“Lymphocyte Activation Gene-3,” “LAG3,” “LAG-3,” or “CD223,” as used herein, refers to a type I transmembrane protein that is expressed on the cell surface of activated CD4+ and CD8+ T cells and subsets of NK and dendritic cells. LAG-3 protein is closely related to CD4, which is a co-receptor for T helper cell activation. Both molecules have four extracellular Ig-like domains and require binding to their ligand, major histocompatibility complex (MHC) class II, for their functional activity. LAG-3 protein is only expressed on the cell surface of activated T cells and its cleavage from the cell surface terminates LAG-3 signaling. LAG-3 can also be found as a soluble protein, which does not bind to MHC class II. LAG-3 also plays an important role in promoting regulatory T cell (Treg) activity and in negatively regulating T cell activation and proliferation. Both natural and induced Treg express increased LAG-3, which is required for their maximal suppressive function. The complete human LAG-3 amino acid sequence can be found under UniProtKB identification number P18627. The human LAG-3 protein is encoded by the human LAG3 gene (NCBI Gene ID: 3902).

“Signal transducer and activator of transcription 1-alpha/beta” or “STAT1,” as used herein, refers to a signal transducer and transcription activator that mediates cellular responses to interferons (IFNs), cytokine KITLG/SCF, and other cytokines and other growth factors. Following type I IFN (IFN-alpha and IFN-beta) binding to cell surface receptors, signaling via protein kinases leads to activation of Jak kinases (TYK2 and JAK1) and to tyrosine phosphorylation of STAT1 and STAT2. The phosphorylated STATs dimerize and associate with ISGF3G/IRF-9 to form a complex termed ISGF3 transcription factor, that enters the nucleus. ISGF3 binds to the IFN stimulated response element (ISRE) to activate the transcription of IFN-stimulated genes (ISG), which drive the cell in an antiviral state. In response to type II IFN (IFN-gamma), STAT1 is tyrosine- and serine-phosphorylated. It then forms a homodimer termed IFN-gamma-activated factor (GAF), migrates into the nucleus and binds to the IFN gamma activated sequence (GAS) to drive the expression of the target genes, inducing a cellular antiviral state. STAT1 becomes activated in response to KITLG/SCF and KIT signaling. STAT1 may also mediate cellular responses to activated FGFR1, FGFR2, FGFR3, and FGFR4. The complete human STAT1 amino acid sequence can be found under UniProtKB identification number P42224. The human STAT1 protein is encoded by the human STAT1 gene (NCBI Gene ID: 6772).

“Cytotoxic T-Lymphocyte Antigen-4” (CTLA-4) refers to an immunoinhibitory receptor belonging to the CD28 family. CTLA-4 is expressed exclusively on T cells in vivo, and binds to two ligands, CD80 and CD86 (also called B7-1 and B7-2, respectively). The term “CTLA-4” as used herein includes human CTLA-4 (hCTLA-4), variants, isoforms, and species homologs of hCTLA-4, and analogs having at least one common epitope with hCTLA-4. The complete hCTLA-4 sequence can be found under GenBank Accession No. AAB59385.

A “subject” includes any human or nonhuman animal. The term “nonhuman animal” includes, but is not limited to, vertebrates such as nonhuman primates, sheep, dogs, and rodents such as mice, rats and guinea pigs. In preferred embodiments, the subject is a human. The terms, “subject” and “patient” are used interchangeably herein.

The use of the term “flat dose” with regard to the methods and dosages of the disclosure means a dose that is administered to a patient without regard for the weight or body surface area (BSA) of the patient. The flat dose is therefore not provided as a mg/kg dose, but rather as an absolute amount of the agent (e.g., the anti-PD-1 antibody). For example, a 60 kg person and a 100 kg person would receive the same dose of an antibody (e.g., 240 mg of an anti-PD-1 antibody).

The use of the term “fixed dose” with regard to a method of the disclosure means that two or more different antibodies in a single composition (e.g., anti-PD-1 antibody and anti-CTLA-4 antibody or an anti-PD-L1 antibody and an anti-CTLA-4 antibody) are present in the composition in particular (fixed) ratios with each other. In some embodiments, the fixed dose is based on the weight (e.g., mg) of the antibodies. In certain embodiments, the fixed dose is based on the concentration (e.g., mg/ml) of the antibodies. In some embodiments, the ratio is at least about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1 mg first antibody (e.g., anti-PD-1 antibody or an anti-PD-L1 antibody) to mg second antibody (e.g., anti-CTLA-4 antibody). For example, the 3:1 ratio of an anti-PD-1 antibody and an anti-CTLA-4 antibody can mean that a vial can contain about 240 mg of the anti-PD-1 antibody and 80 mg of the anti-CTLA-4 antibody or about 3 mg/ml of the anti-PD-1 antibody and 1 mg/ml of the anti-CTLA-4 antibody.

The term “weight-based dose” as referred to herein means that a dose that is administered to a patient is calculated based on the weight of the patient. For example, when a patient with 60 kg body weight requires 3 mg/kg of an anti-PD-1 antibody, one can calculate and use the appropriate amount of the anti-PD-1 antibody (i.e., 180 mg) for administration.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example, an “anti-cancer agent” promotes cancer regression in a subject. In preferred embodiments, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount of an anti-cancer agent preferably inhibits cell growth or tumor growth by at least about 20%, more preferably by at least about 40%, even more preferably by at least about 60%, and still more preferably by at least about 80% relative to untreated subjects. In other preferred embodiments of the disclosure, tumor regression can be observed and continue for a period of at least about 20 days, more preferably at least about 40 days, or even more preferably at least about 60 days. Notwithstanding these ultimate measurements of therapeutic effectiveness, evaluation of immunotherapeutic drugs must also make allowance for immune-related response patterns.

An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell.

An “immune-related response pattern” refers to a clinical response pattern often observed in cancer patients treated with immunotherapeutic agents that produce antitumor effects by inducing cancer-specific immune responses or by modifying native immune processes. This response pattern is characterized by a beneficial therapeutic effect that follows an initial increase in tumor burden or the appearance of new lesions, which in the evaluation of traditional chemotherapeutic agents would be classified as disease progression and would be synonymous with drug failure. Accordingly, proper evaluation of immunotherapeutic agents can require long-term monitoring of the effects of these agents on the target disease.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treatment can be of a subject having a disease or a subject who does not have a disease (e.g., for prophylaxis).

The term “effective dose” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, an increase in overall survival (the length of time from either the date of diagnosis or the start of treatment for a disease, such as cancer, that patients diagnosed with the disease are still alive), or a prevention of impairment or disability due to the disease affliction. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

By way of example, an anti-cancer agent is a drug that promotes cancer regression in a subject. In some embodiments, a therapeutically effective amount of the drug promotes cancer regression to the point of eliminating the cancer. “Promoting cancer regression” means that administering an effective amount of the drug, alone or in combination with an antineoplastic agent, results in a reduction in tumor growth or size, necrosis of the tumor, a decrease in severity of at least one disease symptom, an increase in frequency and duration of disease symptom-free periods, an increase in overall survival, a prevention of impairment or disability due to the disease affliction, or otherwise amelioration of disease symptoms in the patient. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount or dosage of the drug inhibits cell growth or tumor growth by at least about 20%, by at least about 40%, by at least about 60%, or by at least about 80% relative to untreated subjects. In some embodiments, a therapeutically effective amount or dosage of the drug completely inhibits cell growth or tumor growth, i.e., inhibits cell growth or tumor growth by 100%. The ability of a compound to inhibit tumor growth can be evaluated using an assay described herein. Alternatively, this property of a composition can be evaluated by examining the ability of the compound to inhibit cell growth, such inhibition can be measured in vitro by assays known to the skilled practitioner. In some embodiments described herein, tumor regression can be observed and continue for a period of at least about 20 days, at least about 40 days, or at least about 60 days.

The term “biological sample” as used herein refers to biological material isolated from a subject. The biological sample can contain any biological material suitable for determining target gene expression, for example, by sequencing nucleic acids in the tumor (or circulating tumor cells) and identifying a genomic alteration in the sequenced nucleic acids. The biological sample can be any suitable biological tissue or fluid such as, for example, tumor tissue, blood, blood plasma, and serum. In one embodiment, the sample is a tumor tissue biopsy, e.g., a formalin-fixed, paraffin-embedded (FFPE) tumor tissue or a fresh-frozen tumor tissue or the like. In another embodiment, the biological sample is a liquid biopsy that, in some embodiments, comprises one or more of blood, serum, plasma, circulating tumor cells, exoRNA, ctDNA, and cfDNA.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±three days, i.e., every eleven days to every seventeen days. Similar approximations apply, for example, to once about every three weeks, once about every four weeks, once about every five weeks, once about every six weeks, and once about every twelve weeks. In some embodiments, a dosing interval of once about every six weeks or once about every twelve weeks means that the first dose can be administered any day in the first week, and then the next dose can be administered any day in the sixth or twelfth week, respectively. In other embodiments, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

The use of the alternative (e.g., “or”) should be understood to mean either one, both, or any combination thereof of the alternatives. As used herein, the indefinite articles “a” or “an” should be understood to refer to “one or more” of any recited or enumerated component.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one tenth and one hundredth of an integer), unless otherwise indicated.

Abbreviations used herein are defined throughout the present disclosure. A list of additional abbreviations is provided in Table 1.

TABLE 1
List of Abbreviations

| Term | Definition |
|---|---|
| ALK | anaplastic lymphoma kinase |
| AUC | area under the concentration-time curve |
| BSA | body surface area |
| cfDNA | cell-free DNA |
| CI | confidence interval |
| CR | complete response |
| ctDNA | circulating tumor DNA |
| ECOG | Eastern Cooperative Oncology Group |
| EGFR | epidermal growth factor receptor |
| ELISA | enzyme-linked immunosorbent assay |
| exoRNA | exosomal RNA |
| N | number of subjects or observations |
| NCCN | National Comprehensive Cancer Network |
| NSCLC | non-small cell lung cancer |
| ORR | overall response rate |
| RECIST | response evaluation criteria in solid tumors |

Various aspects of the disclosure are described in further detail in the following subsections.

### II. Methods of the Disclosure

The present disclosure is directed to methods of treating a tumor in a human subject, comprising administering to the subject a PD-1 inhibitor, e.g., an anti-PD-1 antibody or anti-PD-L1 antibody, wherein the tumor exhibits a high inflammatory signature score prior to the administration. In some embodiments, the inflammatory signature score is determined by measuring the expression of a panel of inflammatory genes (“inflammatory gene panel”) in a tumor sample obtained from the subject, wherein the inflammatory gene panel comprises CD274 (PD-L1), CD8A, LAG3, and STAT1.

In some embodiments, the inflammatory gene panel consists of less about 20, less than about 19, less than about 18, less than about 17, less than about 16, less than about 15, less than about 14, less than about 13, less than about 12, less than about 11, less than about 10, less than about 9, less than about, less than about 8, less than about 7, less than about 6, or less than about 5 inflammatory genes. In some embodiments, the inflammatory gene panel consists of less than 20 genes. In some embodiments, the inflammatory gene panel consists of less than 19 genes. In some embodiments, the inflammatory gene panel consists of less than 18 genes. In some embodiments, the inflammatory gene panel consists of less than 17 genes. In some embodiments, the inflammatory gene panel consists of less than 16 genes. In some embodiments, the inflammatory gene panel consists of less than 15 genes. In some embodiments, the inflammatory gene panel consists of less than 14 genes. In some embodiments, the inflammatory gene panel consists of less than 13 genes. In some embodiments, the inflammatory gene panel consists of less than 12 genes. In some embodiments, the inflammatory gene panel consists of less than 11 genes. In some embodiments, the inflammatory gene panel consists of less than 10 genes. In some embodiments, the inflammatory gene panel consists of less than 9 genes. In some embodiments, the inflammatory gene panel consists of less than 8 genes. In some embodiments, the inflammatory gene panel consists of less than 7 genes. In some embodiments, the inflammatory gene panel consists of less than 6 genes. In some embodiments, the inflammatory gene panel consists of less than 5 genes. In certain embodiments, the inflammatory gene panel consists of 4 genes. In some embodiments, the inflammatory gene panel consists essentially of CD274 (PD-L1), CD8A, LAG3, and STAT1. In some embodiments, the inflammatory gene panel consists of CD274 (PD-L1), CD8A, LAG3, and STAT1.

In some embodiments, the inflammatory gene panel consists essentially of (or consists of) (i) CD274 (PD-L1) and CD8A, and (ii) 2 additional inflammatory genes, 3 additional inflammatory genes, 4 additional inflammatory genes, 5 additional inflammatory genes, 6 additional inflammatory genes, 7 additional inflammatory genes, 8 additional inflammatory genes, 9 additional inflammatory genes, 10 additional inflammatory genes, 11 additional inflammatory genes, 12 additional inflammatory genes, 13 additional inflammatory genes, 14 additional inflammatory genes, 15 additional inflammatory genes, 16 additional inflammatory genes, or 17 additional inflammatory genes. In some embodiments, the inflammatory gene panel consists essentially of (or consists of) (i) CD274 (PD-L1) and LAG3, and (ii) 2 additional inflammatory genes, 3 additional inflammatory genes, 4 additional inflammatory genes, 5 additional inflammatory genes, 6 additional inflammatory genes, 7 additional inflammatory genes, 8 additional inflammatory genes, 9 additional inflammatory genes, 10 additional inflammatory genes, 11 additional inflammatory genes, 12 additional inflammatory genes, 13 additional inflammatory genes, 14 additional inflammatory genes, 15 additional inflammatory genes, 16 additional inflammatory genes, or 17 additional inflammatory genes. In some embodiments, the inflammatory gene panel consists essentially of (or consists of) (i) CD274 (PD-L1) and STAT1, and (ii) 2 additional inflammatory genes, 3 additional inflammatory genes, 4 additional inflammatory genes, 5 additional inflammatory genes, 6 additional inflammatory genes, 7 additional inflammatory genes, 8 additional inflammatory genes, 9 additional inflammatory genes, 10 additional inflammatory genes, 11 additional inflammatory genes, 12 additional inflammatory genes, 13 additional inflammatory genes, 14 additional inflammatory genes, 15 additional inflammatory genes, 16 additional inflammatory genes, or 17 additional inflammatory genes.

In some embodiments, the inflammatory gene panel consists essentially of (or consists of) (i) CD8A and LAG3, and (ii) 2 additional inflammatory genes, 3 additional inflammatory genes, 4 additional inflammatory genes, 5 additional inflammatory genes, 6 additional inflammatory genes, 7 additional inflammatory genes, 8 additional inflammatory genes, 9 additional inflammatory genes, 10 additional inflammatory genes, 11 additional inflammatory genes, 12 additional inflammatory genes, 13 additional inflammatory genes, 14 additional inflammatory genes, 15 additional inflammatory genes, 16 additional inflammatory genes, or 17 additional inflammatory genes. In some embodiments, the inflammatory gene panel consists essentially of (or consists of) (i) CD8A and STAT1, and (ii) 2 additional inflammatory genes, 3 additional inflammatory genes, 4 additional inflammatory genes, 5 additional inflammatory genes, 6 additional inflammatory genes, 7 additional inflammatory genes, 8 additional inflammatory genes, 9 additional inflammatory genes, 10 additional inflammatory genes, 11 additional inflammatory genes, 12 additional inflammatory genes, 13 additional inflammatory genes, 14 additional inflammatory genes, 15 additional inflammatory genes, 16 additional inflammatory genes, or 17 additional inflammatory genes.

In some embodiments, the inflammatory gene panel consists essentially of (or consists of) (i) LAG3 and STAT1, and (ii) 2 additional inflammatory genes, 3 additional inflammatory genes, 4 additional inflammatory genes, 5 additional inflammatory genes, 6 additional inflammatory genes, 7 additional inflammatory genes, 8 additional inflammatory genes, 9 additional inflammatory genes, 10 additional inflammatory genes, 11 additional inflammatory genes, 12 additional inflammatory genes, 13 additional inflammatory genes, 14 additional inflammatory genes, 15 additional inflammatory genes, 16 additional inflammatory genes, or 17 additional inflammatory genes.

In some embodiments, the inflammatory gene panel consists essentially of (or consists of) (i) CD274 (PD-L1), CD8A, and LAG3, and (ii) 1 additional inflammatory gene, 2 additional inflammatory genes, 3 additional inflammatory genes, 4 additional inflammatory genes, 5 additional inflammatory genes, 6 additional inflammatory genes, 7 additional inflammatory genes, 8 additional inflammatory genes, 9 additional inflammatory genes, 10 additional inflammatory genes, 11 additional inflammatory genes, 12 additional inflammatory genes, 13 additional inflammatory genes, 14 additional inflammatory genes, 15 additional inflammatory genes, or 16 additional inflammatory genes.

In some embodiments, the inflammatory gene panel consists essentially of (or consists of) (i) CD274 (PD-L1), CD8A, and STAT1, and (ii) 1 additional inflammatory gene, 2 additional inflammatory genes, 3 additional inflammatory genes, 4 additional inflammatory genes, 5 additional inflammatory genes, 6 additional inflammatory genes, 7 additional inflammatory genes, 8 additional inflammatory genes, 9 additional inflammatory genes, 10 additional inflammatory genes, 11 additional inflammatory genes, 12 additional inflammatory genes, 13 additional inflammatory genes, 14 additional inflammatory genes, 15 additional inflammatory genes, or 16 additional inflammatory genes.

In some embodiments, the inflammatory gene panel consists essentially of (or consists of) (i) CD274 (PD-L1), LAG3, and STAT1, and (ii) 1 additional inflammatory gene, 2 additional inflammatory genes, 3 additional inflammatory genes, 4 additional inflammatory genes, 5 additional inflammatory genes, 6 additional inflammatory genes, 7 additional inflammatory genes, 8 additional inflammatory genes, 9 additional inflammatory genes, 10 additional inflammatory genes, 11 additional inflammatory genes, 12 additional inflammatory genes, 13 additional inflammatory genes, 14 additional inflammatory genes, 15 additional inflammatory genes, or 16 additional inflammatory genes.

In some embodiments, the inflammatory gene panel consists essentially of (or consists of) (i) CD274 CD8A, LAG3, and STAT1, and (ii) 1 additional inflammatory gene, 2 additional inflammatory genes, 3 additional inflammatory genes, 4 additional inflammatory genes, 5 additional inflammatory genes, 6 additional inflammatory genes, 7 additional inflammatory genes, 8 additional inflammatory genes, 9 additional inflammatory genes, 10 additional inflammatory genes, 11 additional inflammatory genes, 12 additional inflammatory genes, 13 additional inflammatory genes, 14 additional inflammatory genes, 15 additional inflammatory genes, or 16 additional inflammatory genes.

In some embodiments, the inflammatory gene panel consists essentially of (or consists of) (i) CD274 (PD-L1), CD8A, LAG3, and STAT1, and (ii) 1 additional inflammatory gene. In some the inflammatory gene panel consists essentially of (or consists of) (i) CD274 (PD-L1), CD8A, LAG3, and STAT1, and (ii) 2 additional inflammatory genes. In some the inflammatory gene panel consists essentially of (or consists of) (i) CD274 (PD-L1), CD8A, LAG3, and STAT1, and (ii) 3 additional inflammatory genes. In some the inflammatory gene panel consists essentially of (or consists of) (i) CD274 (PD-L1), CD8A, LAG3, and STAT1, and (ii) 4 additional inflammatory genes. In some the inflammatory gene panel consists essentially of (or consists of) (i) CD274 (PD-L1), CD8A, LAG3, and STAT1, and (ii) 5 additional inflammatory genes. In some the inflammatory gene panel consists essentially of (i) CD274 (PD-L1), CD8A, LAG3, and STAT1, and (ii) 6 additional inflammatory genes. In some the inflammatory gene panel consists essentially of (or consists of) (i) CD274 (PD-L1), CD8A, LAG3, and STAT1, and (ii) 7 additional inflammatory genes. In some the inflammatory gene panel consists essentially of (i) CD274 (PD-L1), CD8A, LAG3, and STAT1, and (ii) 8 additional inflammatory genes. In some the inflammatory gene panel consists essentially of (or consists of) (i) CD274 (PD-L1), CD8A, LAG3, and STAT1, and (ii) 9 additional inflammatory genes. In some the inflammatory gene panel consists essentially of (or consists of) (i) CD274 (PD-L1), CD8A, LAG3, and STAT1, and (ii) 10 additional inflammatory genes. In some the inflammatory gene panel consists essentially of (or consists of) (i) CD274 (PD-L1), CD8A, LAG3, and STAT1, and (ii) 11 additional inflammatory genes. In some the inflammatory gene panel consists essentially of (or consists of) (i) CD274 (PD-L1), CD8A, LAG3, and STAT1, and (ii) 12 additional inflammatory genes. In some the inflammatory gene panel consists essentially of (or consists of) (i) CD274 (PD-L1), CD8A, LAG3, and STAT1, and (ii) 13 additional inflammatory genes. In some the inflammatory gene panel consists essentially of (or consists of) (i) CD274 (PD-L1), CD8A, LAG3, and STAT1, and (ii) 14 additional inflammatory genes. In some the inflammatory gene panel consists essentially of (i) CD274 (PD-L1), CD8A, LAG3, and STAT1, and (ii) 15 additional inflammatory genes.

Various genes associated with inflammation are known in the art and can be included in the inflammatory gene panel disclosed herein. For example, the additional inflammatory gene can be selected from the group consisting of CCL2, CCL3, CCL4, CCL5, CCR5, CD27, CD274, CD276, CMKLR1, CXCL10, CXCL11, CXCL9, CXCR6, GZMA, GZMK, HLA-DMA, HLA-DMB, HLA-DOA, HLA-DOB, HLA-DQA1, HLA-DRA, HLA-DRB1, HLA-E, ICOS, IDO1, IFNG, IRF1, NKG7, PDCD1LG2, PRF1, PSMB10, TIGIT, and any combination thereof

In some embodiments, the inflammatory gene panel consists essentially of CD274 (PD-L1), CD8A, LAG3, and STAT1. In some embodiments, the inflammatory gene panel consists of CD274 (PD-L1), CD8A, LAG3, and STAT1.

### II.A. Inflammatory Signature Score

The inflammatory signature score, as used herein, is a measurement of the combined expression level the genes present in the inflammatory gene panel, e.g., comprising, consisting essentially of, or consisting of CD274 (PD-L1), CD8A, LAG3, and STAT1, in a sample obtained from the subject. Any biological sample comprising one or more tumor cell can be used in the methods disclosed herein. In some embodiments, the sample is selected from a tumor biopsy, a blood sample, a serum sample, or any combination thereof. In certain embodiments, the sample is a tumor biopsy collected from the subject prior to administration of the anti-PD-1 antibody. In particular embodiments, the sample obtained from the subject is a formalin-fixed tumor biopsy. In some embodiments, the sample obtained from the subject is a paraffin-embedded tumor biopsy. In some embodiments, the sample obtained from the subject is a fresh-frozen tumor biopsy.

Any method known in the art for measuring the expression of a particular gene or a panel of genes can be used in the methods of the present disclosure. In some embodiments, the expression of one or more of the inflammatory genes in the inflammatory gene panel is determined by detecting the presence of mRNA transcribed from the inflammatory gene, the presence of a protein encoded by the inflammatory gene, or both.

In some embodiments, the expression of one or more of the inflammatory genes is determined by measuring the level of inflammatory gene mRNA, e.g., by measuring the level of one or more of LAG3 mRNA, PD-L1 mRNA, CD8A mRNA, and STAT1 mRNA, in a sample obtained from the subject. In certain embodiments, the inflammatory gene score is determined by measuring the level of LAG3 mRNA, PD-L1 mRNA, CD8A mRNA, and STAT1 mRNA in a sample obtained from the subject. Any method known in the art can be used to measure the level of the inflammatory gene mRNA. In some embodiments, the inflammatory gene mRNA is measured using reverse transcriptase PCR. In some embodiments, the inflammatory gene mRNA is measured using RNA in situ hybridization.

In some embodiments, the expression of one or more of the inflammatory genes is determined by measuring the level of inflammatory gene protein, e.g., by measuring the level of one or more of PD-L1, CD8A, LAG-3, and STAT1, in a sample obtained from the subject. In certain embodiments, the inflammatory gene score is determined by measuring the level of PD-L1, CD8A, LAG-3, and STAT1 in a sample obtained from the subject. Any method known in the art can be used to measure the level of the inflammatory gene protein. In some embodiments, the inflammatory gene protein is measured using an immunohistochemistry (IHC) assay. In certain embodiments, the IHC is an automated IHC.

In some embodiments, the expression of one or more of the inflammatory genes of the inflammatory gene panel is normalized relative to the expression of one or more housekeeping genes. In some embodiments, the one or more housekeeping genes are made up of genes that have relatively consistent expression across various tumor types in various subjects.

In some embodiments, raw gene expression values are normalized following standard gene expression profiling (GEP) protocols. In these embodiments, gene expression signature scores can be calculated as the median or average of the log 2-transformed normalized and scaled expression values across all of the target genes in the signature, and presented on a linear scale. In certain embodiments, scores have positive or negative values, depending on whether gene expression is up- or down-regulated under a particular condition.

In certain embodiments, a high inflammatory signature score is characterized by an inflammatory signature score that is greater than a reference inflammatory signature score. In some embodiments, the reference inflammatory signature score is an average inflammatory signature score. In some embodiments, the average inflammatory signature score is determined by measuring the expression of the genes present in the inflammatory gene panel in tumor samples obtained from a population of subjects, and calculating the average for the population of subjects. In some embodiments, each member of the population of subjects is afflicted with the same tumor as the subject being administered the anti-PD-1 antibody, the anti-PD-L1 antibody, the anti-CTLA-4 antibody, or any combination thereof. In particular embodiments, the average inflammatory signature score is about −0.07, about −0.06, −0.05, about −0.04, about −0.03, or about −0.02. In particular embodiments, the average inflammatory signature score is about −0.04. In certain embodiments, the average inflammatory signature score is about −0.0434.

In some embodiments, a high inflammatory score is characterized by an inflammatory signature score that is at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 125%, at least about 150%, at least about 175%, at least about 200%, at least about 225%, at least about 250%, at least about 275%, or at least about 300% higher than an average inflammatory signature score. In certain embodiments, a high inflammatory score is characterized by an inflammatory signature score that is at least about 25% higher than an average inflammatory signature score. In certain embodiments, a high inflammatory score is characterized by an inflammatory signature score that is at least about 30% higher than an average inflammatory signature score. In certain embodiments, a high inflammatory score is characterized by an inflammatory signature score that is at least about 35% higher than an average inflammatory signature score. In certain embodiments, a high inflammatory score is characterized by an inflammatory signature score that is at least about 40% higher than an average inflammatory signature score. In certain embodiments, a high inflammatory score is characterized by an inflammatory signature score that is at least about 45% higher than an average inflammatory signature score. In certain embodiments, a high inflammatory score is characterized by an inflammatory signature score that is at least about 50% higher than an average inflammatory signature score. In certain embodiments, a high inflammatory score is characterized by an inflammatory signature score that is at least about 55% higher than an average inflammatory signature score. In certain embodiments, a high inflammatory score is characterized by an inflammatory signature score that is at least about 60% higher than an average inflammatory signature score. In certain embodiments, a high inflammatory score is characterized by an inflammatory signature score that is at least about 65% higher than an average inflammatory signature score. In certain embodiments, a high inflammatory score is characterized by an inflammatory signature score that is at least about 70% higher than an average inflammatory signature score. In certain embodiments, a high inflammatory score is characterized by an inflammatory signature score that is at least about 75% higher than an average inflammatory signature score. In certain embodiments, a high inflammatory score is characterized by an inflammatory signature score that is at least about 80% higher than an average inflammatory signature score. In certain embodiments, a high inflammatory score is characterized by an inflammatory signature score that is at least about 85% higher than an average inflammatory signature score. In certain embodiments, a high inflammatory score is characterized by an inflammatory signature score that is at least about 90% higher than an average inflammatory signature score. In certain embodiments, a high inflammatory score is characterized by an inflammatory signature score that is at least about 95% higher than an average inflammatory signature score. In certain embodiments, a high inflammatory score is characterized by an inflammatory signature score that is at least about 100% higher than an average inflammatory signature score. In certain embodiments, a high inflammatory score is characterized by an inflammatory signature score that is at least about 125% higher than an average inflammatory signature score. In certain embodiments, a high inflammatory score is characterized by an inflammatory signature score that is at least about 150% higher than an average inflammatory signature score. In certain embodiments, a high inflammatory score is characterized by an inflammatory signature score that is at least about 175% higher than an average inflammatory signature score. In certain embodiments, a high inflammatory score is characterized by an inflammatory signature score that is at least about 200% higher than an average inflammatory signature score. In certain embodiments, a high inflammatory score is characterized by an inflammatory signature score that is at least about 225% higher than an average inflammatory signature score. In certain embodiments, a high inflammatory score is characterized by an inflammatory signature score that is at least about 250% higher than an average inflammatory signature score. In certain embodiments, a high inflammatory score is characterized by an inflammatory signature score that is at least about 275% higher than an average inflammatory signature score. In certain embodiments, a high inflammatory score is characterized by an inflammatory signature score that is at least about 300% higher than an average inflammatory signature score.

In some embodiments, a high inflammatory score is characterized by an inflammatory signature score that is at least about 1.25-fold, at least about 1.30-fold, at least about 1.35-fold, at least about 1.40-fold, at least about 1.45-fold, at least about 1.50-fold, at least about 1.55-fold, at least about 1.60-fold, at least about 1.65-fold, at least about 1.70-fold, at least about 1.75-fold, at least about 1.80-fold, at least about 1.85-fold, at least about 1.90-fold, at least about 1.95-fold, at least about 2-fold, at least about 2.25-fold, at least about 2.50-fold, at least about 2.75-fold, at least about 3-fold, at least about 3.25-fold, at least about 3.50-fold, at least about 3.75-fold, or at least about 400-fold higher than an average inflammatory signature score. In certain embodiments, a high inflammatory score is characterized by an inflammatory signature score that is at least about 1.25-fold higher than an average inflammatory signature score. In certain embodiments, a high inflammatory score is characterized by an inflammatory signature score that is at least about 1.30-fold higher than an average inflammatory signature score. In certain embodiments, a high inflammatory score is characterized by an inflammatory signature score that is at least about 1.35-fold higher than an average inflammatory signature score. In certain embodiments, a high inflammatory score is characterized by an inflammatory signature score that is at least about 1.40-fold higher than an average inflammatory signature score. In certain embodiments, a high inflammatory score is characterized by an inflammatory signature score that is at least about 1.45-fold higher than an average inflammatory signature score. In certain embodiments, a high inflammatory score is characterized by an inflammatory signature score that is at least about 1.50-fold higher than an average inflammatory signature score. In certain embodiments, a high inflammatory score is characterized by an inflammatory signature score that is at least about 1.55-fold higher than an average inflammatory signature score. In certain embodiments, a high inflammatory score is characterized by an inflammatory signature score that is at least about 1.60-fold higher than an average inflammatory signature score. In certain embodiments, a high inflammatory score is characterized by an inflammatory signature score that is at least about 1.65-fold higher than an average inflammatory signature score. In certain embodiments, a high inflammatory score is characterized by an inflammatory signature score that is at least about 1.70-fold higher than an average inflammatory signature score. In certain embodiments, a high inflammatory score is characterized by an inflammatory signature score that is at least about 1.75-fold higher than an average inflammatory signature score. In certain embodiments, a high inflammatory score is characterized by an inflammatory signature score that is at least about 1.80-fold higher than an average inflammatory signature score. In certain embodiments, a high inflammatory score is characterized by an inflammatory signature score that is at least about 1.85-fold higher than an average inflammatory signature score. In certain embodiments, a high inflammatory score is characterized by an inflammatory signature score that is at least about 1.90-fold higher than an average inflammatory signature score. In certain embodiments, a high inflammatory score is characterized by an inflammatory signature score that is at least about 1.95-fold higher than an average inflammatory signature score. In certain embodiments, a high inflammatory score is characterized by an inflammatory signature score that is at least about 2-fold higher than an average inflammatory signature score. In certain embodiments, a high inflammatory score is characterized by an inflammatory signature score that is at least about 2.25-fold higher than an average inflammatory signature score. In certain embodiments, a high inflammatory score is characterized by an inflammatory signature score that is at least about 2.50-fold higher than an average inflammatory signature score. In certain embodiments, a high inflammatory score is characterized by an inflammatory signature score that is at least about 2.75-fold higher than an average inflammatory signature score. In certain embodiments, a high inflammatory score is characterized by an inflammatory signature score that is at least about 3-fold higher than an average inflammatory signature score. In certain embodiments, a high inflammatory score is characterized by an inflammatory signature score that is at least about 3.25-fold higher than an average inflammatory signature score. In certain embodiments, a high inflammatory score is characterized by an inflammatory signature score that is at least about 3.50-fold higher than an average inflammatory signature score. In certain embodiments, a high inflammatory score is characterized by an inflammatory signature score that is at least about 3.75-fold higher than an average inflammatory signature score. In certain embodiments, a high inflammatory score is characterized by an inflammatory signature score that is at least about 4-fold higher than an average inflammatory signature score.

In certain embodiments, a high inflammatory signature score is characterized by an inflammatory signature score of at least about 0.5, wherein the inflammatory signature score is determined according to a method disclosed herein. In some embodiments, a high inflammatory signature score is characterized by an inflammatory signature score of at least about 0.75, wherein the inflammatory signature score is determined according to a method disclosed herein. In some embodiments, a high inflammatory signature score is characterized by an inflammatory signature score of at least about 1.0, wherein the inflammatory signature score is determined according to a method disclosed herein. In some embodiments, a high inflammatory signature score is characterized by an inflammatory signature score of at least about 1.25, wherein the inflammatory signature score is determined according to a method disclosed herein. In some embodiments, a high inflammatory signature score is characterized by an inflammatory signature score of at least about 1.50, wherein the inflammatory signature score is determined according to a method disclosed herein. In some embodiments, a high inflammatory signature score is characterized by an inflammatory signature score of at least about 1.75, wherein the inflammatory signature score is determined according to a method disclosed herein. In some embodiments, a high inflammatory signature score is characterized by an inflammatory signature score of at least about 2.0, wherein the inflammatory signature score is determined according to a method disclosed herein. In some embodiments, a high inflammatory signature score is characterized by an inflammatory signature score of at least about 2.25, wherein the inflammatory signature score is determined according to a method disclosed herein. In some embodiments, a high inflammatory signature score is characterized by an inflammatory signature score of at least about 2.5, wherein the inflammatory signature score is determined according to a method disclosed herein. In some embodiments, a high inflammatory signature score is characterized by an inflammatory signature score of at least about 2.75, wherein the inflammatory signature score is determined according to a method disclosed herein. In some embodiments, a high inflammatory signature score is characterized by an inflammatory signature score of at least about 3.0, wherein the inflammatory signature score is determined according to a method disclosed herein.

### II.B. Antibodies

The present disclosure is directed to methods for treating a human subject afflicted with a cancer comprising administering to the subject a PD-1 inhibitor, e.g., an anti-PD-1 antibody or an anti-PD-L1 antibody. In some embodiments, the subject is administered an anti-PD-1 monotherapy, e.g., wherein the subject is not administered one or more additional anti-cancer agent. In some embodiments, the subject is administered a combination therapy, e.g., wherein the subject is administered an anti-PD-1 antibody and one or more additional anti-cancer agents. In certain embodiments, the subject is administered a combination therapy comprising an anti-PD-1 antibody and an anti-CTLA-4 antibody.

In other aspects of the present disclosure, an anti-PD-L1 antibody is substituted for the anti-PD-1 antibody. In certain embodiments, the methods comprise administering an anti-PD-L1 antibody to a subject. In some embodiments, the subject is administered an anti-PD-L1 monotherapy. In some embodiments, the subject is administered a combination therapy comprising an anti-PD-L1 antibody and a second anti-cancer agent, e.g., an anti-CTLA-4 antibody.

### II.B.1. Anti-PD-1 Antibodies Useful for the Disclosure

Anti-PD-1 antibodies that are known in the art can be used in the presently described compositions and methods. Various human monoclonal antibodies that bind specifically to PD-1 with high affinity have been disclosed in U.S. Pat. No. 8,008,449. Anti-PD-1 human antibodies disclosed in U.S. Pat. No. 8,008,449 have been demonstrated to exhibit one or more of the following characteristics: (a) bind to human PD-1 with a K_{D} of 1×10⁻⁷ M or less, as determined by surface plasmon resonance using a Biacore biosensor system; (b) do not substantially bind to human CD28, CTLA-4 or ICOS; (c) increase T-cell proliferation in a Mixed Lymphocyte Reaction (MLR) assay; (d) increase interferon-7 production in an MLR assay; (e) increase IL-2 secretion in an MLR assay; (f) bind to human PD-1 and cynomolgus monkey PD-1; (g) inhibit the binding of PD-L1 and/or PD-L2 to PD-1; (h) stimulate antigen-specific memory responses; (i) stimulate antibody responses; and (j) inhibit tumor cell growth in vivo. Anti-PD-1 antibodies usable in the present disclosure include monoclonal antibodies that bind specifically to human PD-1 and exhibit at least one, in some embodiments, at least five, of the preceding characteristics.

Other anti-PD-1 monoclonal antibodies have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

In some embodiments, the anti-PD-1 antibody is selected from the group consisting of nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; see WO 2015/112900), MEDI-0680 (AstraZeneca; also known as AMP-514; see WO 2012/145493), cemiplimab (Regeneron; also known as REGN-2810; see WO 2015/112800), JS001 (TAIZHOU JUNSHI PHARMA; also known as toripalimab; see Si-Yang Liu et al., J. Hematol. Oncol. 10:136 (2017)), BGB-A317 (Beigene; also known as Tislelizumab; see WO 2015/35606 and US 2015/0079109), INCSHR1210 (Jiangsu Hengrui Medicine; also known as SHR-1210; see WO 2015/085847; Si-Yang Liu et al., J. Hematol. Oncol. 10:136 (2017)), TSR-042 (Tesaro Biopharmaceutical; also known as ANB011; see WO2014/179664), GLS-010 (Wuxi/Harbin Gloria Pharmaceuticals; also known as WBP3055; see Si-Yang Liu et al., J. Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), BCD-100 (Biocad; Kaplon et al., mAbs 10(2):183-203 (2018), and IBI308 (Innovent; see WO 2017/024465, WO 2017/025016, WO 2017/132825, and WO 2017/133540).

In one embodiment, the anti-PD-1 antibody is nivolumab. Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56).

In another embodiment, the anti-PD-1 antibody is pembrolizumab. Pembrolizumab is a humanized monoclonal IgG4 (S228P) antibody directed against human cell surface receptor PD-1 (programmed death-1 or programmed cell death-1). Pembrolizumab is described, for example, in U.S. Pat. Nos. 8,354,509 and 8,900,587.

Anti-PD-1 antibodies usable in the disclosed compositions and methods also include isolated antibodies that bind specifically to human PD-1 and cross-compete for binding to human PD-1 with any anti-PD-1 antibody disclosed herein, e.g., nivolumab (see, e.g., U.S. Pat. Nos. 8,008,449 and 8,779,105; WO 2013/173223). In some embodiments, the anti-PD-1 antibody binds the same epitope as any of the anti-PD-1 antibodies described herein, e.g., nivolumab. The ability of antibodies to cross-compete for binding to an antigen indicates that these monoclonal antibodies bind to the same epitope region of the antigen and sterically hinder the binding of other cross-competing antibodies to that particular epitope region. These cross-competing antibodies are expected to have functional properties very similar those of the reference antibody, e.g., nivolumab, by virtue of their binding to the same epitope region of PD-1. Cross-competing antibodies can be readily identified based on their ability to cross-compete with nivolumab in standard PD-1 binding assays such as Biacore analysis, ELISA assays or flow cytometry (see, e.g., WO 2013/173223).

In certain embodiments, the antibodies that cross-compete for binding to human PD-1 with, or bind to the same epitope region of human PD-1 antibody, nivolumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-1 antibodies usable in the compositions and methods of the disclosed disclosure also include antigen-binding portions of the above antibodies. It has been amply demonstrated that the antigen-binding function of an antibody can be performed by fragments of a full-length antibody.

Anti-PD-1 antibodies suitable for use in the disclosed compositions and methods are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and up-regulating the immune system. In certain embodiments, the anti-PD-1 antibody or antigen-binding portion thereof cross-competes with nivolumab for binding to human PD-1.

In some embodiments, the anti-PD-1 antibody is administered at a dose ranging from 0.1 mg/kg to 20.0 mg/kg body weight once every 2, 3, 4, 5, 6, 7, or 8 weeks, e.g., 0.1 mg/kg to 10.0 mg/kg body weight once every 2, 3, or 4 weeks. In other embodiments, the anti-PD-1 antibody is administered at a dose of about 2 mg/kg, about 3 mg/kg, about 4 mg/kg, about 5 mg/kg, about 6 mg/kg, about 7 mg/kg, about 8 mg/kg, about 9 mg/kg, or 10 mg/kg body weight once every 2 weeks. In other embodiments, the anti-PD-1 antibody is administered at a dose of about 2 mg/kg, about 3 mg/kg, about 4 mg/kg, about 5 mg/kg, about 6 mg/kg, about 7 mg/kg, about 8 mg/kg, about 9 mg/kg, or 10 mg/kg body weight once every 3 weeks. In one embodiment, the anti-PD-1 antibody is administered at a dose of about 5 mg/kg body weight about once every 3 weeks. In another embodiment, the anti-PD-1 antibody, e.g., nivolumab, is administered at a dose of about 3 mg/kg body weight about once every 2 weeks. In other embodiments, the anti-PD-1 antibody, e.g., pembrolizumab, is administered at a dose of about 2 mg/kg body weight about once every 3 weeks.

The anti-PD-1 antibody useful for the present disclosure can be administered as a flat dose. In some embodiments, the anti-PD-1 antibody is administered at a flat dose of from about 100 to about 1000 mg, from about 100 mg to about 900 mg, from about 100 mg to about 800 mg, from about 100 mg to about 700 mg, from about 100 mg to about 600 mg, from about 100 mg to about 500 mg, from about 200 mg to about 1000 mg, from about 200 mg to about 900 mg, from about 200 mg to about 800 mg, from about 200 mg to about 700 mg, from about 200 mg to about 600 mg, from about 200 mg to about 500 mg, from about 200 mg to about 480 mg, or from about 240 mg to about 480 mg, In one embodiment, the anti-PD-1 antibody is administered as a flat dose of at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 520 mg, at least about 540 mg, at least about 550 mg, at least about 560 mg, at least about 580 mg, at least about 600 mg, at least about 620 mg, at least about 640 mg, at least about 660 mg, at least about 680 mg, at least about 700 mg, or at least about 720 mg at a dosing interval of about 1, 2, 3, 4, 5, 6, 7, 8, 9, or 10 weeks. In another embodiments, the anti-PD-1 antibody is administered as a flat dose of about 200 mg to about 800 mg, about 200 mg to about 700 mg, about 200 mg to about 600 mg, about 200 mg to about 500 mg, at a dosing interval of about 1, 2, 3, or 4 weeks.

In some embodiments, the anti-PD-1 antibody is administered as a flat dose of about 200 mg at about once every 3 weeks. In other embodiments, the anti-PD-1 antibody is administered as a flat dose of about 200 mg at about once every 2 weeks. In other embodiments, the anti-PD-1 antibody is administered as a flat dose of about 240 mg at about once every 2 weeks. In certain embodiments, the anti-PD-1 antibody is administered as a flat dose of about 480 mg at about once every 4 weeks.

In some embodiments, nivolumab is administered at a flat dose of about 240 mg once about every 2 weeks. In some embodiments, nivolumab is administered at a flat dose of about 240 mg once about every 3 weeks. In some embodiments, nivolumab is administered at a flat dose of about 360 mg once about every 3 weeks. In some embodiments, nivolumab is administered at a flat dose of about 480 mg once about every 4 weeks.

In some embodiments, pembrolizumab is administered at a flat dose of about 200 mg once about every 2 weeks. In some embodiments, pembrolizumab is administered at a flat dose of about 200 mg once about every 3 weeks. In some embodiments, pembrolizumab is administered at a flat dose of about 400 mg once about every 4 weeks.

In some aspects, the PD-1 inhibitor is a small molecule. In some aspects, the PD-1 inhibitor comprises a millamolecule. In some aspects, the PD-1 inhibitor comprises a macrocyclic peptide. In certain aspects, the PD-1 inhibitor comprises BMS-986189. In some aspects, the PD-1 inhibitor comprises an inhibitor disclosed in International Publication No. WO2014/151634, which is incorporated by reference herein in its entirety. In some aspects, the PD-1 inhibitor comprises INCMGA00012 (Incyte Corporation). In some aspects, the PD-1 inhibitor comprises a combination of an anti-PD-1 antibody disclosed herein and a PD-1 small molecule inhibitor.

### II.B.2. Anti-PD-L1 Antibodies Useful for the Disclosure

In certain embodiments, an anti-PD-L1 antibody is substituted for the anti-PD-1 antibody in any of the methods disclosed herein. Anti-PD-L1 antibodies that are known in the art can be used in the compositions and methods of the present disclosure. Examples of anti-PD-L1 antibodies useful in the compositions and methods of the present disclosure include the antibodies disclosed in U.S. Pat. No. 9,580,507. Anti-PD-L1 human monoclonal antibodies disclosed in U.S. Pat. No. 9,580,507 have been demonstrated to exhibit one or more of the following characteristics: (a) bind to human PD-L1 with a K_{D} of 1×10⁻⁷ M or less, as determined by surface plasmon resonance using a Biacore biosensor system; (b) increase T-cell proliferation in a Mixed Lymphocyte Reaction (MLR) assay; (c) increase interferon-7 production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present disclosure include monoclonal antibodies that bind specifically to human PD-L1 and exhibit at least one, in some embodiments, at least five, of the preceding characteristics.

In certain embodiments, the anti-PD-L1 antibody is selected from the group consisting of BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31(suppl):3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), BGB-A333 (BeiGene; see Desai et al., JCO 36 (15suppl):TPS3113 (2018)), and CK-301 (Checkpoint Therapeutics; see Gorelik et al., AACR:Abstract 4606 (April 2016)).

In certain embodiments, the PD-L1 antibody is atezolizumab (TECENTRIQ®). Atezolizumab is a fully humanized IgG1 monoclonal anti-PD-L1 antibody.

In certain embodiments, the PD-L1 antibody is durvalumab (IMFINZI™) Durvalumab is a human IgG1 kappa monoclonal anti-PD-L1 antibody.

In certain embodiments, the PD-L1 antibody is avelumab (BAVENCIO®). Avelumab is a human IgG1 lambda monoclonal anti-PD-L1 antibody.

Anti-PD-L1 antibodies usable in the disclosed compositions and methods also include isolated antibodies that bind specifically to human PD-L1 and cross-compete for binding to human PD-L1 with any anti-PD-L1 antibody disclosed herein, e.g., atezolizumab, durvalumab, and/or avelumab. In some embodiments, the anti-PD-L1 antibody binds the same epitope as any of the anti-PD-L1 antibodies described herein, e.g., atezolizumab, durvalumab, and/or avelumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., atezolizumab and/or avelumab, by virtue of their binding to the same epitope region of PD-L1. Cross-competing antibodies can be readily identified based on their ability to cross-compete with atezolizumab and/or avelumab in standard PD-L1 binding assays such as Biacore analysis, ELISA assays or flow cytometry (see, e.g., WO 2013/173223).

In certain embodiments, the antibodies that cross-compete for binding to human PD-L1 with, or bind to the same epitope region of human PD-L1 antibody as, atezolizumab, durvalumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies usable in the compositions and methods of the disclosed disclosure also include antigen-binding portions of the above antibodies. It has been amply demonstrated that the antigen-binding function of an antibody can be performed by fragments of a full-length antibody.

Anti-PD-L1 antibodies suitable for use in the disclosed compositions and methods are antibodies that bind to PD-L1 with high specificity and affinity, block the binding of PD-1, and inhibit the immunosuppressive effect of the PD-1 signaling pathway. In any of the compositions or methods disclosed herein, an anti-PD-L1 “antibody” includes an antigen-binding portion or fragment that binds to PD-L1 and exhibits the functional properties similar to those of whole antibodies in inhibiting receptor binding and up-regulating the immune system. In certain embodiments, the anti-PD-L1 antibody or antigen-binding portion thereof cross-competes with atezolizumab, durvalumab, and/or avelumab for binding to human PD-L1.

The anti-PD-L1 antibody useful for the present disclosure can be any PD-L1 antibody that specifically binds to PD-L1, e.g., antibodies that cross-compete with durvalumab, avelumab, or atezolizumab for binding to human PD-1, e.g., an antibody that binds to the same epitope as durvalumab, avelumab, or atezolizumab. In a particular embodiment, the anti-PD-L1 antibody is durvalumab. In other embodiments, the anti-PD-L1 antibody is avelumab. In some embodiments, the anti-PD-L1 antibody is atezolizumab.

In some embodiments, the anti-PD-L1 antibody is administered at a dose ranging from about 0.1 mg/kg to about 20.0 mg/kg body weight, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg, about once every 2, 3, 4, 5, 6, 7, or 8 weeks.

In some embodiments, the anti-PD-L1 antibody is administered at a dose of about 15 mg/kg body weight at about once every 3 weeks. In other embodiments, the anti-PD-L1 antibody is administered at a dose of about 10 mg/kg body weight at about once every 2 weeks.

In other embodiments, the anti-PD-L1 antibody useful for the present disclosure is a flat dose. In some embodiments, the anti-PD-L1 antibody is administered as a flat dose of from about 200 mg to about 1600 mg, about 200 mg to about 1500 mg, about 200 mg to about 1400 mg, about 200 mg to about 1300 mg, about 200 mg to about 1200 mg, about 200 mg to about 1100 mg, about 200 mg to about 1000 mg, about 200 mg to about 900 mg, about 200 mg to about 800 mg, about 200 mg to about 700 mg, about 200 mg to about 600 mg, about 700 mg to about 1300 mg, about 800 mg to about 1200 mg, about 700 mg to about 900 mg, or about 1100 mg to about 1300 mg. In some embodiments, the anti-PD-L1 antibody is administered as a flat dose of at least about 240 mg, at least about 300 mg, at least about 320 mg, at least about 400 mg, at least about 480 mg, at least about 500 mg, at least about 560 mg, at least about 600 mg, at least about 640 mg, at least about 700 mg, at least 720 mg, at least about 800 mg, at least about 840 mg, at least about 880 mg, at least about 900 mg, at least 960 mg, at least about 1000 mg, at least about 1040 mg, at least about 1100 mg, at least about 1120 mg, at least about 1200 mg, at least about 1280 mg, at least about 1300 mg, at least about 1360 mg, or at least about 1400 mg, at a dosing interval of about 1, 2, 3, or 4 weeks. In some embodiments, the anti-PD-L1 antibody is administered as a flat dose of about 1200 mg at about once every 3 weeks. In other embodiments, the anti-PD-L1 antibody is administered as a flat dose of about 800 mg at about once every 2 weeks. In other embodiments, the anti-PD-L1 antibody is administered as a flat dose of about 840 mg at about once every 2 weeks.

In some embodiments, atezolizumab is administered as a flat dose of about 1200 mg once about every 3 weeks. In some embodiments, atezolizumab is administered as a flat dose of about 800 mg once about every 2 weeks. In some embodiments, atezolizumab is administered as a flat dose of about 840 mg once about every 2 weeks.

In some embodiments, avelumab is administered as a flat dose of about 800 mg once about every 2 weeks.

In some embodiments, durvalumab is administered at a dose of about 10 mg/kg once about every 2 weeks. In some embodiments, durvalumab is administered as a flat dose of about 800 mg/kg once about every 2 weeks. In some embodiments, durvalumab is administered as a flat dose of about 1200 mg/kg once about every 3 weeks.

In some aspects, the PD-L1 inhibitor is a small molecule. In some aspects, the PD-L1 inhibitor comprises a millamolecule. In some aspects, the PD-L1 inhibitor comprises a macrocyclic peptide. In certain aspects, the PD-L1 inhibitor comprises BMS-986189.

In some aspects, the PD-L1 inhibitor comprises a millamolecule having a formula set forth in formula (I):

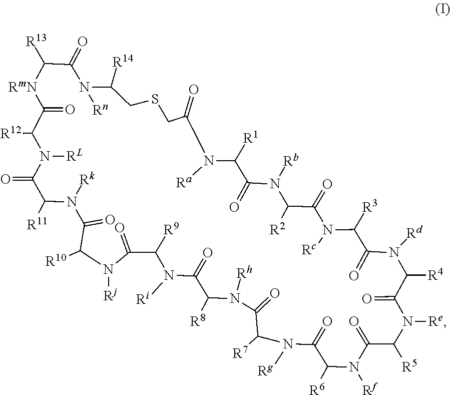

wherein R¹-R¹³ are amino acid side chains, R^{a}-Rⁿ are hydrogen, methyl, or form a ring with a vicinal R group, and R¹⁴ is —C(O)NHR¹⁵, wherein R¹⁵ is hydrogen, or a glycine residue optionally substituted with additional glycine residues and/or tails which can improve pharmacokinetic properties. In some aspects, the PD-L1 inhibitor comprises a compound disclosed in International Publication No. WO2014/151634, which is incorporated by reference herein in its entirety. In some aspects, the PD-L1 inhibitor comprises a compound disclosed in International Publication No. WO2016/039749, WO2016/149351, WO2016/077518, WO2016/100285, WO2016/100608, WO2016/126646, WO2016/057624, WO2017/151830, WO2017/176608, WO2018/085750, WO2018/237153, or WO2019/070643, each of which is incorporated by reference herein in its entirety.

In certain aspects the PD-L1 inhibitor comprises a small molecule PD-L1 inhibitor disclosed in International Publication No. WO2015/034820, WO2015/160641, WO2018/044963, WO2017/066227, WO2018/009505, WO2018/183171, WO2018/118848, WO2019/147662, or WO2019/169123, each of which is incorporated by reference herein in its entirety.

In some aspects, the PD-L1 inhibitor comprises a combination of an anti-PD-L1 antibody disclosed herein and a PD-L1 small molecule inhibitor disclosed herein.

### II.B.3. Anti-CTLA-4 Antibodies

Anti-CTLA-4 antibodies that are known in the art can be used in the compositions and methods of the present disclosure. Anti-CTLA-4 antibodies of the instant disclosure bind to human CTLA-4 so as to disrupt the interaction of CTLA-4 with a human B7 receptor. Because the interaction of CTLA-4 with B7 transduces a signal leading to inactivation of T-cells bearing the CTLA-4 receptor, disruption of the interaction effectively induces, enhances or prolongs the activation of such T cells, thereby inducing, enhancing or prolonging an immune response.

Human monoclonal antibodies that bind specifically to CTLA-4 with high affinity have been disclosed in U.S. Pat. Nos. 6,984,720. Other anti-CTLA-4 monoclonal antibodies have been described in, for example, U.S. Pat. Nos. 5,977,318, 6,051,227, 6,682,736, and 7,034,121 and International Publication Nos. WO 2012/122444, WO 2007/113648, WO 2016/196237, and WO 2000/037504, each of which is incorporated by reference herein in its entirety. The anti-CTLA-4 human monoclonal antibodies disclosed in U.S. Pat. No. 6,984,720 have been demonstrated to exhibit one or more of the following characteristics: (a) binds specifically to human CTLA-4 with a binding affinity reflected by an equilibrium association constant (Kₐ) of at least about 10⁷ M⁻¹, or about 10⁹ M⁻¹, or about 10¹⁰ M⁻¹ to 10¹¹ M⁻¹ or higher, as determined by Biacore analysis; (b) a kinetic association constant (kₐ) of at least about 10³, about 10⁴, or about 10⁵ m⁻¹ s⁻¹; (c) a kinetic disassociation constant (k_{d}) of at least about 10³, about 10⁴, or about 10⁵ m⁻¹ s⁻¹; and (d) inhibits the binding of CTLA-4 to B7-1 (CD80) and B7-2 (CD86). Anti-CTLA-4 antibodies useful for the present disclosure include monoclonal antibodies that bind specifically to human CTLA-4 and exhibit at least one, at least two, or at least three of the preceding characteristics.

In certain embodiments, the CTLA-4 antibody is selected from the group consisting of ipilimumab (also known as YERVOY®, MDX-010, 10D1; see U.S. Pat. No. 6,984,720), MK-1308 (Merck), AGEN-1884 (Agenus Inc.; see WO 2016/196237), and tremelimumab (AstraZeneca; also known as ticilimumab, CP-675,206; see WO 2000/037504 and Ribas, Update Cancer Ther. 2(3): 133-39 (2007)). In particular embodiments, the anti-CTLA-4 antibody is ipilimumab.

In particular embodiments, the CTLA-4 antibody is ipilimumab for use in the compositions and methods disclosed herein. Ipilimumab is a fully human, IgG1 monoclonal antibody that blocks the binding of CTLA-4 to its B7 ligands, thereby stimulating T cell activation and improving overall survival (OS) in patients with advanced melanoma.

In particular embodiments, the CTLA-4 antibody is tremelimumab.

In particular embodiments, the CTLA-4 antibody is MK-1308.

In particular embodiments, the CTLA-4 antibody is AGEN-1884.

Anti-CTLA-4 antibodies usable in the disclosed compositions and methods also include isolated antibodies that bind specifically to human CTLA-4 and cross-compete for binding to human CTLA-4 with any anti-CTLA-4 antibody disclosed herein, e.g., ipilimumab and/or tremelimumab. In some embodiments, the anti-CTLA-4 antibody binds the same epitope as any of the anti-CTLA-4 antibodies described herein, e.g., ipilimumab and/or tremelimumab. The ability of antibodies to cross-compete for binding to an antigen indicates that these antibodies bind to the same epitope region of the antigen and sterically hinder the binding of other cross-competing antibodies to that particular epitope region. These cross-competing antibodies are expected to have functional properties very similar those of the reference antibody, e.g., ipilimumab and/or tremelimumab, by virtue of their binding to the same epitope region of CTLA-4. Cross-competing antibodies can be readily identified based on their ability to cross-compete with ipilimumab and/or tremelimumab in standard CTLA-4 binding assays such as Biacore analysis, ELISA assays or flow cytometry (see, e.g., WO 2013/173223).

In certain embodiments, the antibodies that cross-compete for binding to human CTLA-4 with, or bind to the same epitope region of human CTLA-4 antibody as, ipilimumab and/or tremelimumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-CTLA-4 antibodies usable in the compositions and methods of the disclosed disclosure also include antigen-binding portions of the above antibodies. It has been amply demonstrated that the antigen-binding function of an antibody can be performed by fragments of a full-length antibody.

Anti-CTLA-4 antibodies suitable for use in the disclosed methods or compositions are antibodies that bind to CTLA-4 with high specificity and affinity, block the activity of CTLA-4, and disrupt the interaction of CTLA-4 with a human B7 receptor. In any of the compositions or methods disclosed herein, an anti-CTLA-4 “antibody” includes an antigen-binding portion or fragment that binds to CTLA-4 and exhibits the functional properties similar to those of whole antibodies in inhibiting the interaction of CTLA-4 with a human B7 receptor and up-regulating the immune system. In certain embodiments, the anti-CTLA-4 antibody or antigen-binding portion thereof cross-competes with ipilimumab and/or tremelimumab for binding to human CTLA-4.

In some embodiments, the anti-CTLA-4 antibody or antigen-binding portion thereof is administered at a dose ranging from 0.1 mg/kg to 10.0 mg/kg body weight once every 2, 3, 4, 5, 6, 7, or 8 weeks. In some embodiments, the anti-CTLA-4 antibody or antigen-binding portion thereof is administered at a dose of 1 mg/kg or 3 mg/kg body weight once every 3, 4, 5, or 6 weeks. In one embodiment, the anti-CTLA-4 antibody or antigen-binding portion thereof is administered at a dose of 3 mg/kg body weight once every 2 weeks. In another embodiment, the anti-PD-1 antibody or antigen-binding portion thereof is administered at a dose of 1 mg/kg body weight once every 6 weeks.

In some embodiments, the anti-CTLA-4 antibody or antigen-binding portion thereof is administered as a flat dose. In some embodiments, the anti-CTLA-4 antibody is administered at a flat dose of from about 10 to about 1000 mg, from about 10 mg to about 900 mg, from about 10 mg to about 800 mg, from about 10 mg to about 700 mg, from about 10 mg to about 600 mg, from about 10 mg to about 500 mg, from about 100 mg to about 1000 mg, from about 100 mg to about 900 mg, from about 100 mg to about 800 mg, from about 100 mg to about 700 mg, from about 100 mg to about 100 mg, from about 100 mg to about 500 mg, from about 100 mg to about 480 mg, or from about 240 mg to about 480 mg. In one embodiment, the anti-CTLA-4 antibody or antigen-binding portion thereof is administered as a flat dose of at least about 60 mg, at least about 80 mg, at least about 100 mg, at least about 120 mg, at least about 140 mg, at least about 160 mg, at least about 180 mg, at least about 200 mg, at least about 220 mg, at least about 240 mg, at least about 260 mg, at least about 280 mg, at least about 300 mg, at least about 320 mg, at least about 340 mg, at least about 360 mg, at least about 380 mg, at least about 400 mg, at least about 420 mg, at least about 440 mg, at least about 460 mg, at least about 480 mg, at least about 500 mg, at least about 520 mg at least about 540 mg, at least about 550 mg, at least about 560 mg, at least about 580 mg, at least about 600 mg, at least about 620 mg, at least about 640 mg, at least about 660 mg, at least about 680 mg, at least about 700 mg, or at least about 720 mg. In another embodiment, the anti-CTLA-4 antibody or antigen-binding portion thereof is administered as a flat dose about once every 1, 2, 3, 4, 5, 6, 7, or 8 weeks.

In some embodiments, ipilimumab is administered at a dose of about 3 mg/kg once about every 3 weeks. In some embodiments, ipilimumab is administered at a dose of about 10 mg/kg once about every 3 weeks. In some embodiments, ipilimumab is administered at a dose of about 10 mg/kg once about every 12 weeks. In some embodiments, the ipilimumab is administered for four doses.

### II.B.4. Combination Therapies

In certain embodiments, the anti-PD-1 antibody, the anti-PD-L1 antibody, and/or the anti-CTLA-4 antibody are administered at a therapeutically effective amount. In some embodiments, the method comprises administering a therapeutically effective amount of anti-PD-1 antibody and an anti-CTLA-4 antibody. In other embodiments, the method comprises administering a therapeutically effective amount of anti-PD-L1 antibody and an anti-CTLA-4 antibody. Any anti-PD-1, anti-PD-L1, or anti-CTLA-4 antibody disclosed herein can be used in the method. In certain embodiments, the anti-PD-1 antibody comprises nivolumab. In some embodiments, the anti-PD-1 antibody comprises pembrolizumab. In some embodiments, the anti-PD-L1 antibody comprises atezolizumab. In some embodiments, the anti-PD-L1 antibody comprises durvalumab. In some embodiments, the anti-PD-L1 antibody comprises avelumab. In some embodiments, the anti-CTLA-4 antibody comprises ipilimumab. In some embodiments, the anti-CTLA-4 antibody comprises ipilimumab tremelimumab.

In some embodiments, the (a) anti-PD-1 antibody or the anti-PD-L1 antibody and the (b) anti-CTLA-4 antibody are each administered once about every 2 weeks, once about every 3 weeks, once about every 4 weeks, once about every 5 weeks, or once about every 6 weeks. In some embodiments, the anti-PD-1 antibody or the anti-PD-L1 antibody is administered once about every 2 weeks, once about every 3 weeks or once about every 4 weeks, and the anti-CTLA-4 antibody is administered once about every 6 weeks. In some embodiments, the anti-PD-1 antibody or anti-PD-L1 antibody is administered on the same day as the anti-CTLA-4 antibody. In some embodiments, the anti-PD-1 antibody or the anti-PD-L1 antibody is administered on a different day than the anti-CTLA-4 antibody.

In some embodiments, the anti-CTLA-4 antibody is administered at a dose ranging from about 0.1 mg/kg to about 20.0 mg/kg body weight once about every 2, 3, 4, 5, 6, 7, or 8 weeks. In some embodiments, the anti-CTLA-4 antibody is administered at a dose of about 0.1 mg/kg, about 0.3 mg/kg, about 0.6 mg/kg, about 0.9 mg/kg, about 1 mg/kg, about 3 mg/kg, about 6 mg/kg, about 9 mg/kg, about 10 mg/kg, about 12 mg/kg, about 15 mg/kg, about 18 mg/kg, or about 20 mg/kg. In certain embodiments, the anti-CTLA-4 antibody is administered at a dose of about 1 mg/kg once about every 4 weeks. In some embodiments, the anti-CTLA-4 antibody is administered at a dose of about 1 mg/kg once about every 6 weeks.

In some embodiments, the anti-CTLA-4 antibody is administered at a flat dose. In some embodiments, the anti-CTLA-4 antibody is administered at a flat dose ranging from at least about 40 mg to at least about 1600 mg. In some embodiments, the anti-CTLA-4 antibody is administered at a flat dose of at least about 40 mg, at least about 50 mg, at least about 60 mg, at least about 70 mg, at least about 80 mg, at least about 90 mg, at least about 100 mg, at least about 110 mg, at least about 120 mg, at least about 130 mg, at least about 140 mg, at least about 150 mg, at least about 160 mg, at least about 170 mg, at least about 180 mg, at least about 190 mg, or at least about 200 mg. In some embodiments, the CTLA-4 antibody is administered at a flat dose of at least about 220 mg, at least about 230 mg, at least about 240 mg, at least about 250 mg, at least about 260 mg, at least about 270 mg, at least about 280 mg, at least about 290 mg, at least about 300 mg, at least about 320 mg, at least about 360 mg, at least about 400 mg, at least about 440 mg, at least about 480 mg, at least about 520 mg, at least about 560 mg, or at least about 600 mg. In some embodiments, the CTLA-4 antibody is administered at a flat dose of at least about 640 mg, at least about 720 mg, at least about 800 mg, at least about 880 mg, at least about 960 mg, at least about 1040 mg, at least about 1120 mg, at least about 1200 mg, at least about 1280 mg, at least about 1360 mg, at least about 1440 mg, or at least about 1600 mg. In some embodiments, the anti-CTLA-4 antibody is administered in a flat dose at least once about every 2, 3, 4, 5, 6, 7, or 8 weeks.

In certain embodiments, the anti-PD-1 antibody is administered at a dose of about 2 mg/kg once about every 3 weeks and the anti-CTLA-4 antibody is administered at a dose of about 1 mg/kg once about every 6 weeks. In some embodiments, the anti-PD-1 antibody is administered at a dose of about 3 mg/kg once about every 2 weeks and the anti-CTLA-4 antibody is administered at a dose of about 1 mg/kg once about every 6 weeks. In some embodiments, the anti-PD-1 antibody is administered at a dose of about 6 mg/kg once about every 4 weeks and the anti-CTLA-4 antibody is administered at a dose of about 1 mg/kg once about every 6 weeks.

In certain embodiments, the anti-PD-1 antibody is administered at a flat dose of about 200 mg once about every 3 weeks and the anti-CTLA-4 antibody is administered at a dose of about 1 mg/kg once about every 6 weeks. In some embodiments, the anti-PD-1 antibody is administered at a flat dose of about 200 mg once about every 2 weeks and the anti-CTLA-4 antibody is administered at a dose of about 1 mg/kg once about every 6 weeks. In some embodiments, the anti-PD-1 antibody is administered at a flat dose of about 240 mg once about every 2 weeks and the anti-CTLA-4 antibody is administered at a dose of about 1 mg/kg once about every 6 weeks. In some embodiments, the anti-PD-1 antibody is administered at a flat dose of about 480 mg once about every 4 weeks and the anti-CTLA-4 antibody is administered at a dose of about 1 mg/kg once about every 6 weeks.

In certain embodiments, the anti-PD-1 antibody is administered at a flat dose of about 200 mg once about every 3 weeks and the anti-CTLA-4 antibody is administered at a flat dose of about 80 mg once about every 6 weeks. In some embodiments, the anti-PD-1 antibody is administered at a flat dose of about 200 mg once about every 2 weeks and the anti-CTLA-4 antibody is administered at a dose of about 80 mg once about every 6 weeks. In some embodiments, the anti-PD-1 antibody is administered at a flat dose of about 240 mg once about every 2 weeks and the anti-CTLA-4 antibody is administered at a dose of about 80 mg once about every 6 weeks. In some embodiments, the anti-PD-1 antibody is administered at a flat dose of about 480 mg once about every 4 weeks and the anti-CTLA-4 antibody is administered at a dose of about 80 mg once about every 6 weeks.

In certain embodiments, the anti-PD-L1 antibody is administered at a dose of about 10 mg/kg once about every 2 weeks and the anti-CTLA-4 antibody is administered at a dose of about 1 mg/kg once about every 6 weeks. In some embodiments, the anti-PD-L1 antibody is administered at a dose of about 15 mg/kg once about every 3 weeks and the anti-CTLA-4 antibody is administered at a dose of about 1 mg/kg once about every 6 weeks.

In certain embodiments, the anti-PD-L1 antibody is administered at a flat dose of about 800 mg once about every 2 weeks and the anti-CTLA-4 antibody is administered at a dose of about 1 mg/kg once about every 6 weeks. In some embodiments, the anti-PD-L1 antibody is administered at a flat dose of about 1200 mg once about every 3 weeks and the anti-CTLA-4 antibody is administered at a dose of about 1 mg/kg once about every 6 weeks.

In certain embodiments, the anti-PD-L1 antibody is administered at a flat dose of about 800 mg once about every 2 weeks and the anti-CTLA-4 antibody is administered at a flat dose of about 80 mg once about every 6 weeks. In some embodiments, the anti-PD-L1 antibody is administered at a flat dose of about 1200 mg once about every 3 weeks and the anti-CTLA-4 antibody is administered at a dose of about 80 mg once about every 6 weeks.

In some embodiments, the anti-PD-1 antibody, e.g., nivolumab, is administered at a dose of about 3 mg/kg and the anti-CTLA-4 antibody is administered at a dose of about 1 mg/kg on the same day, once about every 3 weeks for 4 doses, then the anti-PD-1 antibody, e.g., nivolumab, is administered at a flat dose of 240 mg once about every 2 weeks or 480 mg once about every 4 weeks. In some embodiments, the anti-PD-1 antibody, e.g., nivolumab, is administered at a dose of about 1 mg/kg and the anti-CTLA-4 antibody is administered at a dose of about 3 mg/kg on the same day, once about every 3 weeks for 4 doses, then the anti-PD-1 antibody, e.g., nivolumab, is administered at a flat dose of 240 mg once about every 2 weeks or 480 mg once about every 4 weeks.

### II.B.5. Additional Anticancer Therapies

In some aspects of the present disclosure, the methods disclosed herein further comprise administering an anti-PD-1 antibody (or an anti-PD-L1 antibody) and an additional anticancer therapy. In certain embodiments, the method comprising administering an anti-PD-1 antibody (or an anti-PD-L1 antibody), an anti-CTLA-4 antibody, and an additional anticancer therapy. The additional anticancer therapy can comprise any therapy known in the art for the treatment of a tumor in a subject and/or any standard-of-care therapy, as disclosed herein. In some embodiments, the additional anticancer therapy comprises a surgery, a radiation therapy, a chemotherapy, an immunotherapy, or any combination thereof. In some embodiments, the additional anticancer therapy comprises a chemotherapy, including any chemotherapy disclosed herein. In some embodiment, the additional anticancer therapy comprises an immunotherapy. In some embodiments, the additional anticancer therapy comprises administration of an antibody or antigen-binding portion thereof that specifically binds LAG-3, TIGIT, TIM3, NKG2a, OX40, ICOS, MICA, CD137, KIR, TGFβ, IL-10, IL-8, B7-H4, Fas ligand, CXCR4, mesothelin, CD27, GITR, or any combination thereof.

### II.C. Tumors

In some embodiments, the tumor is derived from a cancer selected from the group consisting of hepatocellular cancer, gastroesophageal cancer, melanoma, bladder cancer, lung cancer, kidney cancer, head and neck cancer, colon cancer, and any combination thereof. In certain embodiments, the tumor is derived from a hepatocellular cancer, wherein the tumor has a high inflammatory signature score. In certain embodiments, the tumor is derived from a gastroesophageal cancer, wherein the tumor has a high inflammatory signature score. In certain embodiments, the tumor is derived from a melanoma, wherein the tumor has a high inflammatory signature score. In certain embodiments, the tumor is derived from a bladder cancer, wherein the tumor has a high inflammatory signature score. In certain embodiments, the tumor is derived from a lung cancer, wherein the tumor has a high inflammatory signature score. In certain embodiments, the tumor is derived from a kidney cancer, wherein the tumor has a high inflammatory signature score. In certain embodiments, the tumor is derived from a head and neck cancer, wherein the tumor has a high inflammatory signature score. In certain embodiments, the tumor is derived from a colon cancer, wherein the tumor has a high inflammatory signature score.

In certain embodiments, the subject has received one, two, three, four, five or more prior cancer treatments. In other embodiments, the subject is treatment-naïve. In some embodiments, the subject has progressed on other cancer treatments. In certain embodiments, the prior cancer treatment comprised an immunotherapy. In other embodiments, the prior cancer treatment comprised a chemotherapy. In some embodiments, the tumor has reoccurred. In some embodiments, the tumor is metastatic. In other embodiments, the tumor is not metastatic. In some embodiments, the tumor is locally advanced.

In some embodiments, the subject has received a prior therapy to treat the tumor and the tumor is relapsed or refractory. In certain embodiments, the at least one prior therapy comprises a standard-of-care therapy. In some embodiments, the at least one prior therapy comprises a surgery, a radiation therapy, a chemotherapy, an immunotherapy, or any combination thereof. In some embodiments, the at least one prior therapy comprises a chemotherapy. In some embodiments, the subject has received a prior immuno-oncology (I-O) therapy to treat the tumor and the tumor is relapsed or refractory. In some embodiments, the subject has received more than one prior therapy to treat the tumor and the subject is relapsed or refractory. In other embodiments, the subject has received either an anti-PD-1 or anti-PD-L1 antibody therapy.

In some embodiments, the previous line of therapy comprises a chemotherapy. In some embodiments, the chemotherapy comprises a platinum-based therapy. In some embodiments, the platinum-based therapy comprises a platinum-based antineoplastic selected from the group consisting of cisplatin, carboplatin, oxaliplatin, nedaplatin, triplatin tetranitrate, phenanthriplatin, picoplatin, satraplatin, and any combination thereof. In certain embodiments, the platinum-based therapy comprises cisplatin. In one particular embodiment, the platinum-based therapy comprises carboplatin.

In some embodiments, the at least one prior therapy is selected from a therapy comprising administration of an anticancer agent selected from the group consisting of a platinum agent (e.g., cisplatin, carboplatin), a taxanes agent (e.g., paclitaxel, albumin-bound paclitaxel, docetaxel), vinorelbine, vinblastine, etoposide, pemetrexed, gemcitabine, bevacizumab (AVASTIN®), erlotinib (TARCEVA®), crizotinib (XALKORI®), cetuximab (ERBITUX®), and any combination thereof. In certain embodiments, the at least one prior therapy comprises a platinum-based doublet chemotherapy.

In some embodiments, the subject has experienced disease progression after the at least one prior therapy. In certain embodiments, the subject has received at least two prior therapies, at least three prior therapies, at least four prior therapies, or at least five prior therapies. In certain embodiments, the subject has received at least two prior therapies. In one embodiment, the subject has experienced disease progression after the at least two prior therapies. In certain embodiments, the at least two prior therapies comprises a first prior therapy and a second prior therapy, wherein the subject has experienced disease progression after the first prior therapy and/or the second prior therapy, and wherein the first prior therapy comprises a surgery, a radiation therapy, a chemotherapy, an immunotherapy, or any combination thereof, and wherein the second prior therapy comprises a surgery, a radiation therapy, a chemotherapy, an immunotherapy, or any combination thereof. In some embodiments, the first prior therapy comprises a platinum-based doublet chemotherapy, and the second prior therapy comprises a single-agent chemotherapy. In certain embodiments, the single-agent chemotherapy comprises docetaxel.

### II.E. Pharmaceutical Compositions and Dosages

Therapeutic agents of the present disclosure can be constituted in a composition, e.g., a pharmaceutical composition containing an antibody and/or a cytokine and a pharmaceutically acceptable carrier. As used herein, a “pharmaceutically acceptable carrier” includes any and all solvents, dispersion media, coatings, antibacterial and antifungal agents, isotonic and absorption delaying agents, and the like that are physiologically compatible. Preferably, the carrier for a composition containing an antibody is suitable for intravenous, intramuscular, subcutaneous, parenteral, spinal or epidermal administration (e.g., by injection or infusion), whereas the carrier for a composition containing an antibody and/or a cytokine is suitable for non-parenteral, e.g., oral, administration. In some embodiments, the subcutaneous injection is based on Halozyme Therapeutics' ENHANZE® drug-delivery technology (see U.S. Pat. No. 7,767,429, which is incorporated by reference herein in its entirety). ENHANZE® uses a co-formulation of an antibody with recombinant human hyaluronidase enzyme (rHuPH20), which removes traditional limitations on the volume of biologics and drugs that can be delivered subcutaneously due to the extracellular matrix (see U.S. Pat. No. 7,767,429). A pharmaceutical composition of the disclosure can include one or more pharmaceutically acceptable salts, anti-oxidant, aqueous and non-aqueous carriers, and/or adjuvants such as preservatives, wetting agents, emulsifying agents and dispersing agents. Therefore, in some embodiments, the pharmaceutical composition for the present disclosure can further comprise recombinant human hyaluronidase enzyme, e.g., rHuPH20.

In some embodiments, the method comprises administering an anti-PD-1 antibody (or an anti-PD-L1 antibody) and an anti-CTLA-4 antibody, wherein the anti-PD-1 antibody (or the anti-PD-L1 antibody) is administered in a fixed dose with the anti-CTLA-4 antibody in a single composition. In some embodiments, the anti-PD-1 antibody is administered in a fixed dose with the anti-CTLA-4 antibody. In some embodiments, the anti-PD-L1 antibody is administered in a fixed dose with the anti-CTLA-4 antibody in a single composition. In some embodiments, the ratio of the anti-PD-1 antibody (or the anti-PD-L1 antibody) to the anti-CTLA-4 antibody is at least about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1.

Although higher nivolumab monotherapy dosing up to 10 mg/kg every two weeks has been achieved without reaching the maximum tolerated does (MTD), the significant toxicities reported in other trials of checkpoint inhibitors plus anti-angiogenic therapy (see, e.g., Johnson et al., 2013; Rini et al., 2011) support the selection of a nivolumab dose lower than 10 mg/kg.

Treatment is continued as long as clinical benefit is observed or until unacceptable toxicity or disease progression occurs. Nevertheless, in certain embodiments, the dosages of the anti-PD-1 antibody, the anti-PD-L1 antibody, and/or the anti-CTLA-4 antibody administered are significantly lower than the approved dosage, i.e., a subtherapeutic dosage, of the agent. The anti-PD-1 antibody, the anti-PD-L1 antibody, and/or the anti-CTLA-4 antibody can be administered at the dosage that has been shown to produce the highest efficacy as monotherapy in clinical trials, e.g., about 3 mg/kg of nivolumab administered once every three weeks (Topalian et al., 2012a; Topalian et al., 2012), or at a significantly lower dose, i.e., at a subtherapeutic dose.

Dosage and frequency vary depending on the half-life of the antibody in the subject. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present disclosure can be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present disclosure can be administered via one or more routes of administration using one or more of a variety of methods well known in the art. As will be appreciated by the skilled artisan, the route and/or mode of administration will vary depending upon the desired results.

### III. Kits

Also within the scope of the present disclosure are kits comprising (a) an anti-PD-1 antibody or an anti-PD-L1 antibody for therapeutic uses. Kits typically include a label indicating the intended use of the contents of the kit and instructions for use. The term label includes any writing, or recorded material supplied on or with the kit, or which otherwise accompanies the kit. Accordingly, this disclosure provides a kit for treating a subject afflicted with a tumor, the kit comprising: (a) a dosage ranging from 0.1 to 10 mg/kg body weight of an anti-PD-1 antibody or a dosage ranging from 0.1 to 20 mg/kg body weight of an anti-PD-L1 antibody; and (b) instructions for using the anti-PD-1 antibody or the anti-PD-L1 antibody in the methods disclosed herein. This disclosure further provides a kit for treating a subject afflicted with a tumor, the kit comprising: (a) a dosage ranging from about 4 mg to about 500 mg of an anti-PD-1 antibody or a dosage ranging from about 4 mg to about 2000 mg of an anti-PD-L1 antibody; and (b) instructions for using the anti-PD-1 antibody or the anti-PD-L1 antibody in the methods disclosed herein. In some embodiments, this disclosure provides a kit for treating a subject afflicted with a tumor, the kit comprising: (a) a dosage ranging from 200 mg to 800 mg of an anti-PD-1 antibody or a dosage ranging from 200 mg to 1800 mg of an anti-PD-L1 antibody; and (b) instructions for using the anti-PD-1 antibody or the anti-PD-L1 antibody in the methods disclosed herein.

In certain embodiments for treating human patients, the kit comprises an anti-human PD-1 antibody disclosed herein, e.g., nivolumab or pembrolizumab. In certain embodiments for treating human patients, the kit comprises an anti-human PD-L1 antibody disclosed herein, e.g., atezolizumab, durvalumab, or avelumab.

In some embodiments, the kit further comprises an anti-CTLA-4 antibody. In certain embodiments for treating human patients, the kit comprises an anti-human CTLA-4 antibody disclosed herein, e.g., ipilimumab, tremelimumab, MK-1308, or AGEN-1884.

In some embodiments, the kit further includes an inflammatory gene panel assay disclosed herein. In some embodiments, the kit further includes instructions to administer the anti-PD-1 antibody or the anti-PD-L1 antibody to a subject identified as having a high inflammatory signature score, according to the methods disclosed herein. In other embodiments, the kit further includes an anti-CTLA-4 antibody and instructions to administer (a) the anti-PD-1 antibody or the anti-PD-L1 antibody and (b) the anti-CTLA-4 antibody to a subject identified as having a high inflammatory signature score, according to the methods disclosed herein.

All of the references cited above, as well as all references cited herein, are incorporated herein by reference in their entireties.

The following examples are offered by way of illustration and not by way of limitation.

### EXAMPLES

### Example 1: Assessment of Inflammation Biomarkers in Relation to Clinical Outcomes in Nivolumab-Treated Patients with Advanced Hepatocellular Carcinoma

Liver cancer is the fourth leading cause of cancer-related mortality globally, with the majority of liver cancers being hepatocellular carcinoma (HCC). Patients with advanced HCC have few effective treatment options, and agents capable of achieving robust and durable responses remain an unmet need in hepatocellular. Clinical trials for approved first-line and second-line targeted therapies report median overall survivals ranging from 10.7-13.6 months and 10.2-10.6 months, respectively (see, Abou-Alfa et al., N Engl J Med. 379(1):54-63 (2018); Bruix et al., Lancet 389(10064):56-66 (2017); Llovet et al., N Engl J Med. 359(4):378-90 (2008); and Kudo et al., Lancet. 391(10126):1163-73 (2018)). Nivolumab (“NIVO”) binds to PD-1 receptors, which are expressed primarily on activated T cells, and thus prevents binding of the PD-L1 and PD-L2 ligands, which are expressed on tumor cells. Nivolumab has demonstrated durable responses, manageable safety, and long-term survival in patients with advanced HCC, regardless of etiology, with/without prior sorafenib (SOR) treatment in Clinical Trial NCT01658878 (see, El-Khoueiry et al., Lancet. 389:2492-2502 (2017)). NIVO is approved in many countries, including the United States, in SOR-experienced patients with HCC based on results from Clinical Trial NCT01658878.

The present example is directed to findings from exploratory biomarker analyses of nivolumab-treated patients with advanced HCC from Clinical Trial NCT01658878.

### Study Design

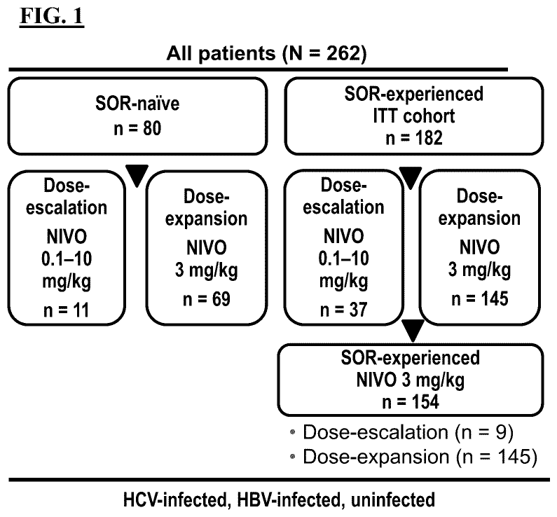

The present data is related to Cohorts 1 and 2 of Clinical Trial NCT01658878, which together had a total of 262 subjects (FIG. 1). Cohort 1 comprised 80 SOR-naïve subjects, and Cohort 2 comprised 182 SOR-experienced subjects. Eleven subjects in Cohort 1 and 37 subjects in Cohort 2 were administered 0.1-10 mg/kg nivolumab as part of a dose-escalation analysis. Sixty-nine subject in Cohort 1 and 145 subjects in cohort 2 were administered 3 mg/kg nivolumab as part of a dose-expansion analysis. Following initial treatment, 154 subjects in Cohort 2 (9 subject from the dose-escalation study and 145 subjects from the dose-expansion study) were administered maintenance nivolumab at 3 mg/kg.

The primary endpoints of Clinical Trial NCT01658878 were safety and tolerability (dose-escalation) as well as objected response rate (ORR; dose-expansion). Secondary endpoints included ORR (dose-escalation), disease control rate, time to response, duration of response, and overall survival. Exploratory endpoints included biomarker assessments, which are discussed here.

Data generated from Clinical Trial NCT01658878, including an ORR of 14.3% and a duration of response (DOR) of at least 12 months in 50% of subjects, contributed to the USFDA approval of nivolumab for the treatment of SOR-experienced patients with HCC.

Eligible subjects had (i) histologically confirmed advanced HCC not amenable to curative resection; (ii) Child-Pugh scores ≤7 (escalation) or ≤6 (expansion); (iii) progression on at least one prior line of systemic therapy or intolerance or refusal of SOR; (iv) AST and ALT≤5×upper limit of normal and bilirubin≤3 mg/dL; (v) for HBV-infected patients, viral load less than 100 IU/mL and concomitant effective antiviral therapy; and (vi) for HCV-infected patients, active or resolved infection as evidenced by detectable HCV RNA or antibody. Subjects were excluded that had any history of hepatic encephalopathy, prior or current clinically significant ascites, or active HBV and HCV co-infection.

Pretreatment tumor samples (fresh or archival) were obtained from patients in the escalation and expansion phases receiving 3 mg/kg nivolumab (saved for IHC) or 0.1-10 mg/kg nivolumab (saved for RNA sequencing).

### Biomarker Assessments

Samples were analyzed using (i) IHC to assess PD-L1, PD-1, T-cell markers (CD3, CD4, CD8, FOXP3), and macrophage markers (CD68, CD163); and (ii) RNA sequencing to assess tumor inflammatory signatures. Biomarkers were assessed for their association with clinical outcomes including BOR by blinded independent review committee (per RECIST v1.1) and overall survival. Analyses were performed using the standard Limma and Cox regression framework.

### Biomarker Analysis

### PD-L1

In the overall population, 195 subjects had evaluable PD-L1 data (SOR-naïve, n=58; SOR-experienced, n=137; Table 2). Clinically meaningful responses were observed in all subjects, including those with PD-L1<1%, and 6 subject had a complete response. In the overall population, numerically higher objective response rates were observed in subjects with PD-L1≥1% versus PD-L1<1% with overlapping 95% confidence intervals. The SOR-experienced population had ORRs comparable to those of the overall population.

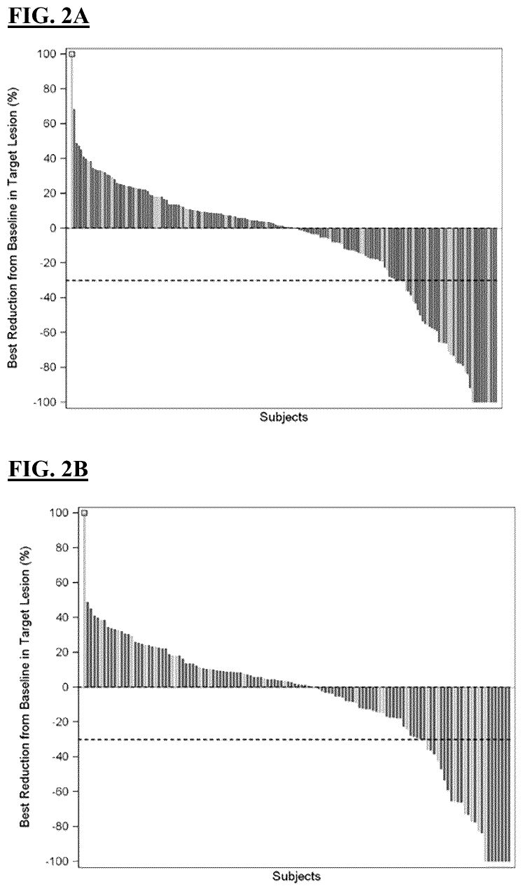

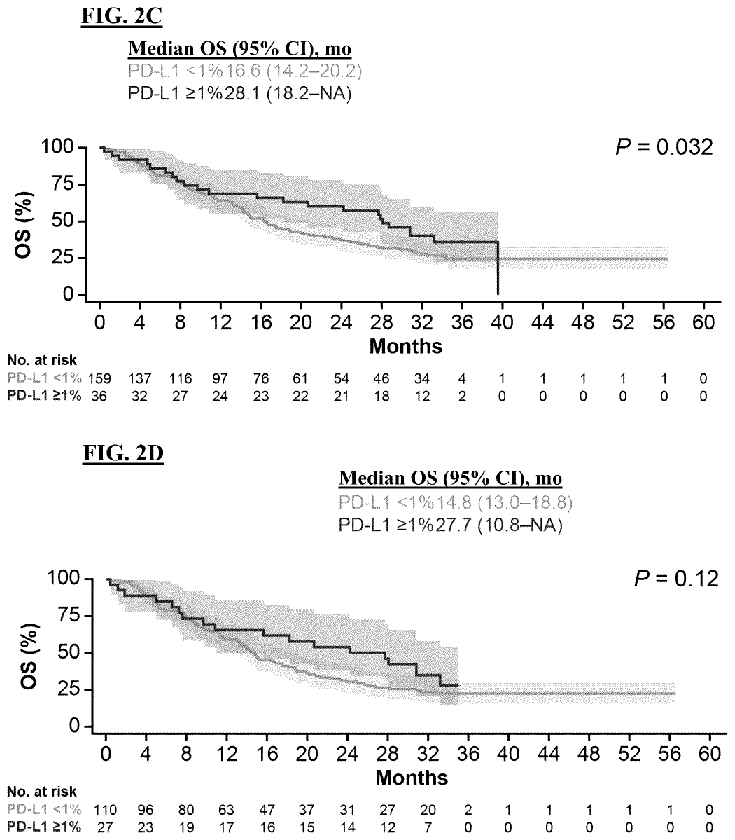

In the overall population, deep responses were observed regardless of PD-L1 status (FIGS. 2A-2B). Tumor cell PD-L1 expression in at least 1% of tumor cells was significantly associated with overall survival (FIG. 2C; P=0.032). In general, positive PD-L1 expression in at least 1% of tumor cells associated with a higher overall survival in SOR-experienced subjects, however this difference was not statistically significant (FIG. 2D)

TABLE 2
Best overall response by tumor cell PD-L1 status.

| | | Overall | | | |
|---|---|---|---|---|---|
| | | population | | | |
| | | (SOR-naïve | | SOR- | |
| | | and SOR- | | experienced | |
| PD-L1 | | experienced) | | population | |
| cutoff | | n = 195 | | n = 137 | |
| PD-L1 | Total, n (%) | 159 | (81.5) | 110 | (80.2) |
| <1% | Objective response rate, | 15.7 | (10.8-22.2) | 12.7 | (7.6-20.3) |
| | % (95% CI) | | | | |
| | Complete response, n (%) | 6 | (3.7) | 4 | (3.6) |
| | Partial response, n (%) | 19 | (11.9) | 10 | (9) |
| | Stable disease, n (%) | 66 | (41.5) | 49 | (44.5) |
| | Progressive disease, n (%) | 59 | (37.1) | 42 | (38.1) |
| PD-L1 | Total, n (%) | 36 | (18.4) | 27 | (19.7) |
| >1% | Objective response rate, | 27.7 | (15.7-44.1) | 25.9 | (12.9-44.9) |
| | % (95% CI) | | | | |
| | Complete response, n (%) | 2 | (5.5) | 1 | (3.7) |
| | Partial response, n (%) | 8 | (22.2) | 6 | (22.2) |
| | Stable disease, n (%) | 9 | (25) | 8 | (29.6) |
| | Progressive disease, n (%) | 15 | (41.6) | 10 | (37) |

Tumor PD-L1 expression was not found to be significantly different when stratified by geographical region (Asians v. non-Asians; data not shown).

### T-Cell Markers

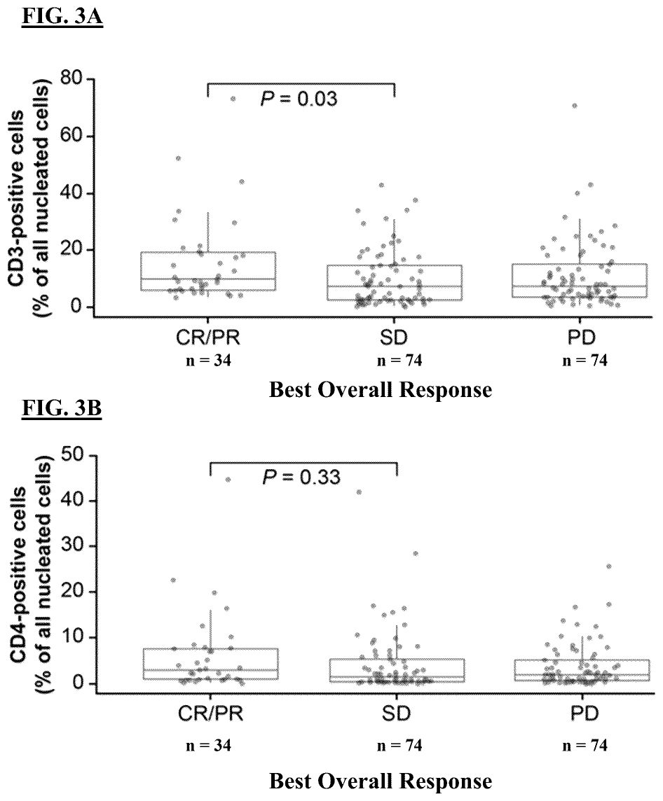

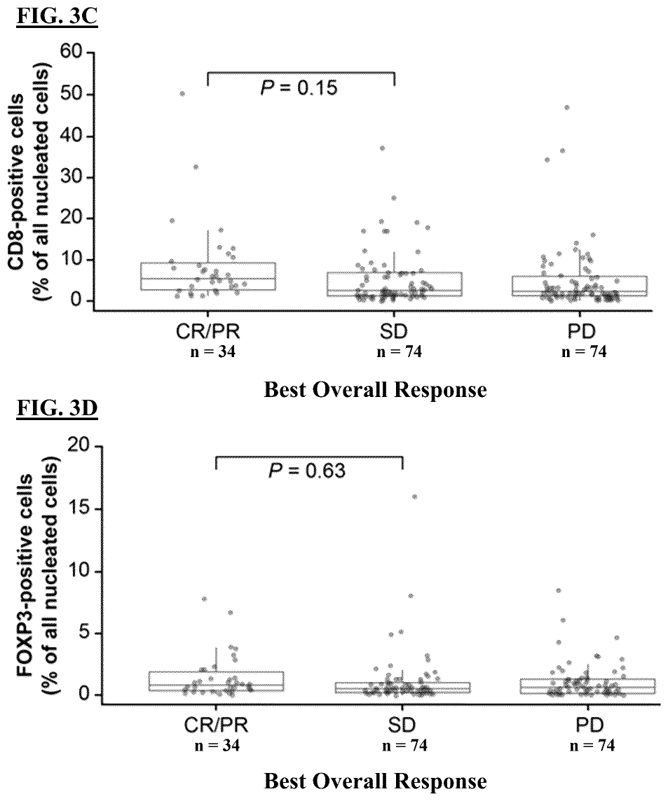

Expression profiles of the T-cell markers CD3, CD8, CD4, and FOX-3 were analyzed in tumor samples obtained from subject prior to administration of nivolumab. CD3-positive cell frequency was observed to be associated with response (CR/PR compared to SD; P=0.03; FIG. 3A). No significant association was observed between CD4-, CD8-, or FOXP3-positive cell frequency and response (FIGS. 3B-3D). In the tumor microenvironment, CD3-positive cell frequency was higher versus the other T-cell markers assessed (data not shown). T-cell marker distribution was not found to be significantly different when stratified by viral etiology (HBV- or HCV-infected, or uninfected; data not shown) or geographical region (Asians v. non-Asians; data not shown).

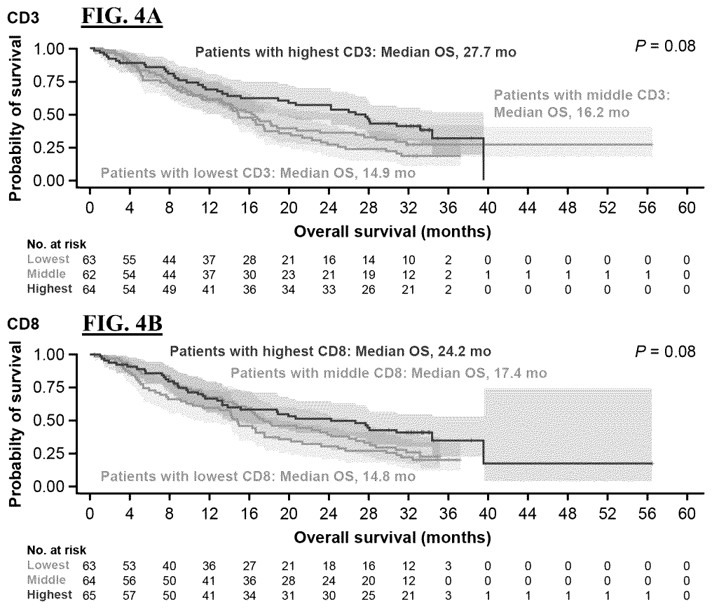

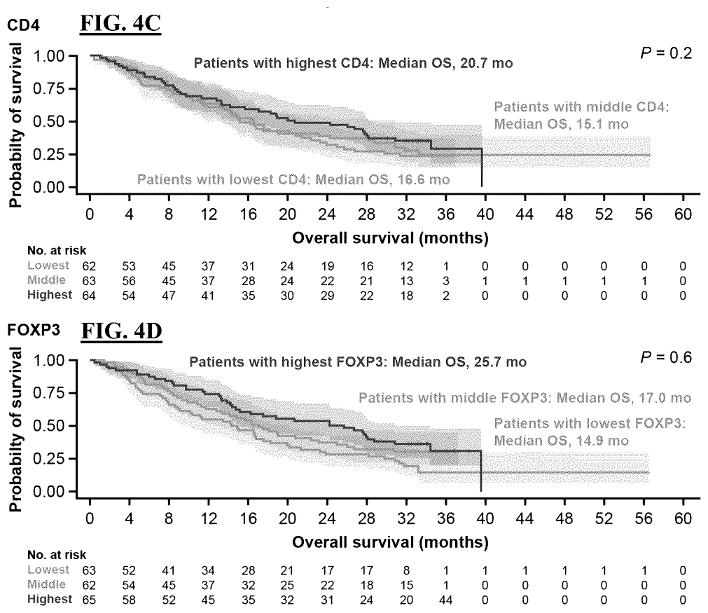

Tumor inflammation, as measured by CD3 or CD8 expression, had a non-significant trend towards improved overall survival (FIGS. 4A-4B; P=0.08), and to a lesser extent for CD4 or FOXP3 expression (FIGS. 4C-4D).

### Macrophage Markers

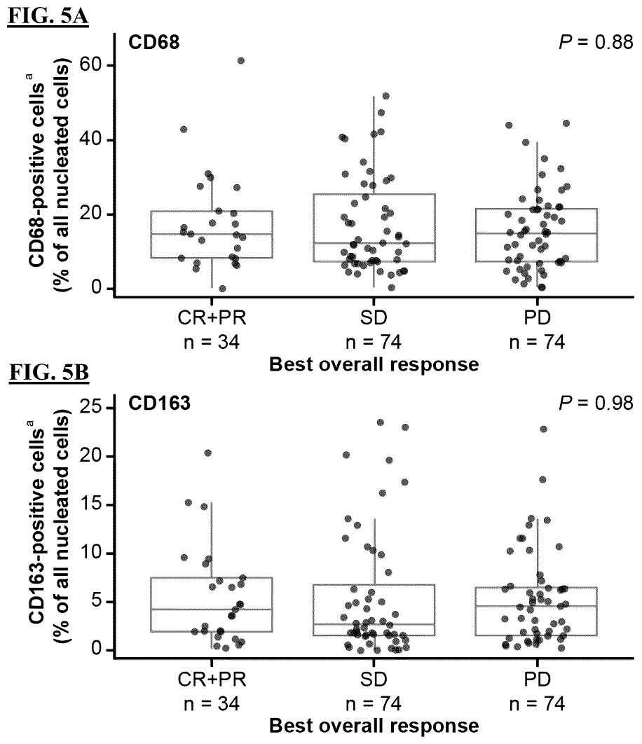

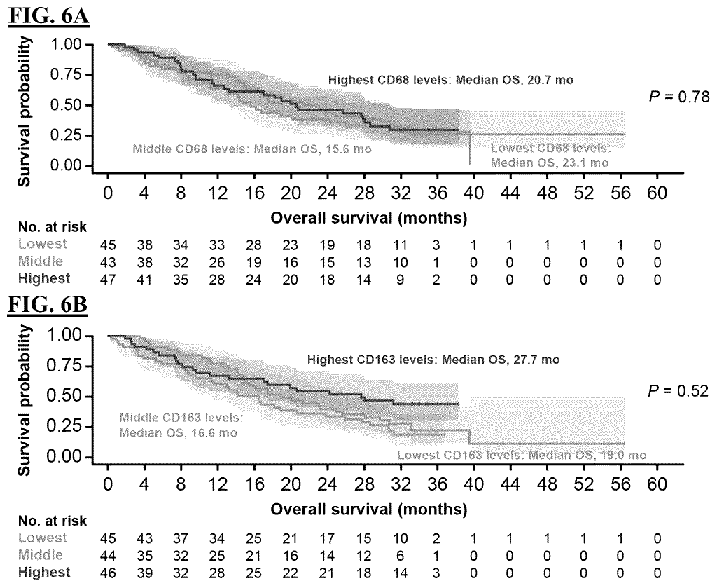

Expression profiles of the macrophage markers CD68 and CD163 were analyzed in tumor samples obtained from subject prior to administration of nivolumab. No association between CD68- and CD163-expression and clinical outcome was observed (FIGS. 5A-5B and FIGS. 6A-6B). In addition, macrophage maker distribution was not found to be significantly different when stratified by viral etiology (HBV- or HCV-infected, or uninfected; data not shown) or geographical region (Asians v. non-Asians; data not shown).

### Tumor Immune Gene Signatures

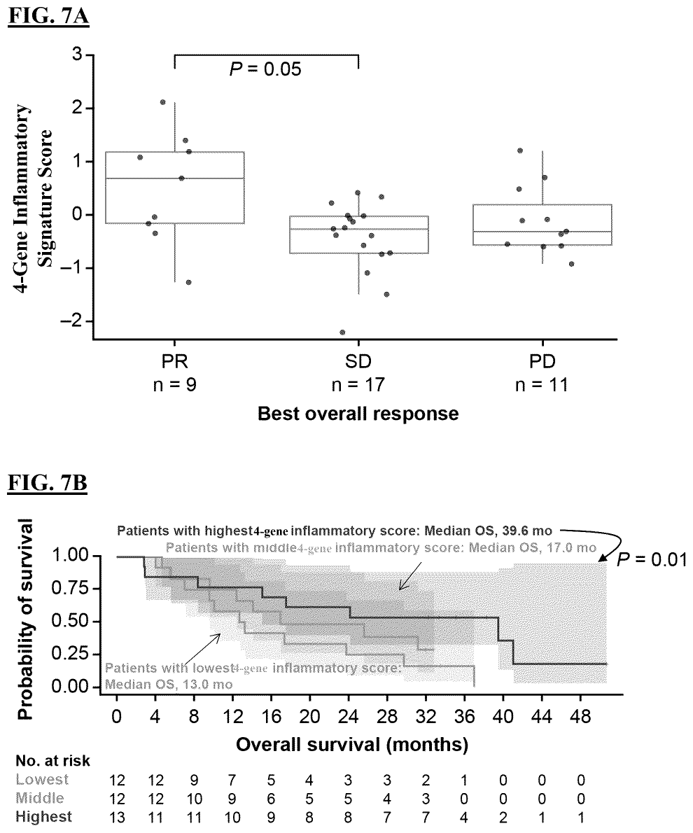

For the subset of subjects for whom data were available (n=37), RNA sequencing was used for gene expression profiling to evaluate tumor immune infiltration and inflammatory signatures (Table 3). In particular, several inflammatory signatures, such as the 4-gene inflammatory signature of the present disclosure (comprising CD274 (PD-L1), CD8A, LAG3, and STAT1), the Gajewski 13-Gene Inflammatory Signature, the Merck 6-gene interferon gamma signature, the NanoString interferon gamma biology signature, and the NanoString T-cell exhaustion signature correlated significantly with improved response and overall survival (Table 3). In particular, the average 4-gene inflammatory signature score, as described herein, was observed to be significantly higher in patients experiencing a partial response as compared to stable disease (p=0.05; FIG. 7A). In addition, the average median 4-gene inflammatory score significantly associated with improved overall survival (p=0.01; FIG. 7B).

TABLE 3
Relationship between tumor immune gene signatures and clinical
response in overall population.

| Immune gene signatures | Genes within | ORR | OS |
|---|---|---|---|
| evaluated | each signature | P-value^{a} | P-value |
| 4-gene Inflammatory | CD274 (PD-L1), CD8A, | 0.05 | 0.01 |
| Signature | LAG3, STAT1 | | |
| Cytolytic Activity | GZMA, PRF1 | 0.1 | 0.2 |
| Signature¹ | | | |
| Gajewski 13-Gene | CCL2, CCL3, CCL4, | 0.04 | 0.05 |
| Inflammatory Signature² | CD8A, CXCL10, CXCL9, | | |
| | GZMK, HLA-DMA, | | |
| | HLA-DMB, HLA-DOA, | | |
| | HLA-DOB, ICOS, IRF1 | | |
| Merck 6-gene Interferon | CXCL10, CXCL9, HLA- | 0.05 | 0.009 |
| Gamma Signature³ | DRA, IDO1, IFNG, | | |
| | STAT1 | | |
| Nano String ® Antigen | CMKLR1, HLA-DQA1, | 0.6 | 0.08 |
| Presenting Cells | HLA-DRB1, PSMB10 | | |
| Signature³ | | | |
| Nano String ® Interferon | CCL5, CD27, CXCL9, | 0.07 | 0.008 |
| Gamma Biology | CXCR6, IDO1, STAT1 | | |
| Signature³ | | | |
| NanoString ® T-cell | CD274 (PD-L1), CD276, | 0.03 | 0.04 |
| Exhaustion Signature³ | CD8A, LAG3, | | |
| | PDCD1LG2, TIGIT | | |
| Nano String ® T/NK | HLA-E, NKG7 | 0.3 | 0.04 |
| Cell Signature³ | | | |
| Ribas 10-gene Interferon | CCR5, CXCL10, | 0.07 | 0.02 |
| Gamma Signature³ | CXCL11, CXCL9, | | |
| | GZMA, HLA-DRA, | | |
| | IDO1, IFNG, PRF1, | | |
| | STAT1 | | |

¹Danilova L, et al. Proc Natl Acad Sci. 2016; 113: E7769-E7777;
²Spranger S, et al. Nature. 2015; 523: 231-235;
³Ayers M, etal. J Clin Invest. 2017; 127: 2930-2940.

The 4-gene inflammatory signature score was not found to be significantly different when stratified by viral etiology (HBV- or HCV-infected, or uninfected; data not shown) or geographical region (Asians v. non-Asians; data not shown).

In Clinical Trial NCT01658878 cohorts 1 & 2, durable responses were observed in both SOR-naïve and SOR-experienced patients regardless of tumor cell PD-L1 status. In this retrospective analysis of pretreatment tumor samples from patients with advanced HCC, tumor cell PD-L1 expression was associated with OS; however, this association was not significant in SOR-experienced patients. CD3⁺ T-cell frequency was associated with response to nivolumab, with a trend towards improved survival with CD3 and CD8 positivity. Higher scores for several inflammatory signatures, including the 4-gene inflammatory signature, were associated with improved response and overall survival.

### Example 2: Association of PD-L1 Combined Positive Score and Immune Gene Signatures with Efficacy of Nivolumab±Ipilimumab in Patients with Metastatic Gastroesophageal Cancer

Combination therapy comprising nivolumab (NIVO) and ipilimumab (IPI) demonstrated clinically meaningful antitumor activity and a manageable safety profile in patients with chemotherapy-refractory gastroesophageal cancer in the phase 1/2 (NCT01928394; Janjigian Y Y, et al. J Clin Oncol. 2018; 36:2836-2844). In the current exploratory analysis from clinical trial NCT01928394, the expression of selected immune gene signatures was evaluated to determine if there is association with efficacy of nivolumab monotherapy of combination therapy with ipilimumab.

### Study Design

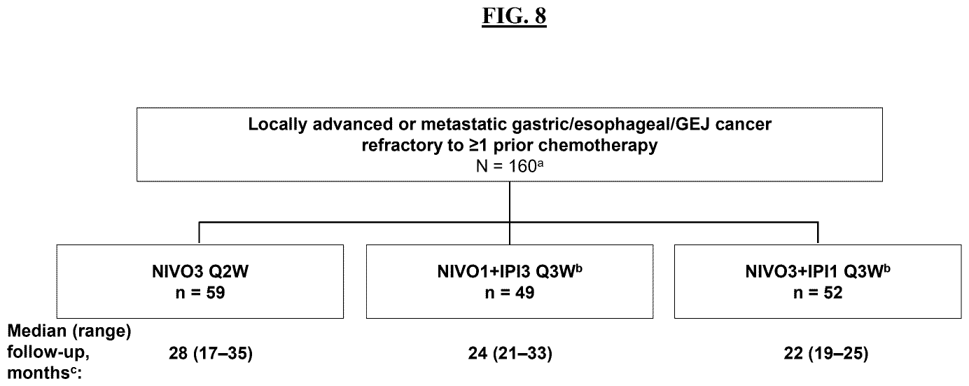

Subjects having locally advanced or metastatic gastric/esophageal/GEJ cancer that was refractory to ≥1 prior chemotherapy were randomly assigned to one of the following: nivolumab 3 mg/kg (NIVO3) intravenously every 2 weeks (n=59); nivolumab 1 mg/kg plus ipilimumab 3 mg/kg (NIVO1+IPI3) every 3 weeks for four cycles (n=49); or nivolumab 3 mg/kg plus ipilimumab 1 mg/kg (NIVO3+IPI1) every 3 weeks for four cycles (n=52) (FIG. 8). All combination regimens were followed by NIVO3 every 2 weeks until disease progression or unacceptable adverse event (AE).

The primary end point was objective response rate (ORR), defined as the best response of complete response or partial response divided by the number of treated patients, per RECIST version 1.1. Secondary end points included overall survival (OS), progression-free survival (PFS), time to response, duration of response (DOR), and safety. Tumor response was assessed using imaging every 6 weeks for 24 weeks, then every 12 weeks until disease progression or treatment discontinuation. Survival was monitored continuously while patients were receiving treatment and every 3 months after treatment discontinuation. Exploratory endpoints included association between tumor PD-L1 expression and efficacy and safety.

Key eligibility criteria for the esophagogastric cancer cohort included diagnosis of locally advanced or metastatic gastric, esophageal, or GEJ adenocarcinoma with disease progression while taking or intolerance of at least one chemotherapy regimen; measurable disease as assessed by Response Evaluation Criteria in Solid Tumors (RECIST) version 1.118; Eastern Cooperative Oncology Group performance status of 0 or 1; and adequate organ function. Patients with human epidermal growth factor receptor 2-positive tumors were eligible if they had received previous treatment with trastuzumab. Key exclusion criteria included suspected autoimmune disease; hepatitis B virus or human immunodeficiency virus infection; conditions requiring corticosteroids or other immunosuppressive medications; and previous immune checkpoint inhibitor therapy.

### Biomarker Analysis

### PD-L1 Expression

Biological samples were collected from subjects prior to immunotherapy, and a subset of subject samples were available for PD-L1 expression analyses (Table 4).

TABLE 4
Baseline characteristics and response: overall and PD-L1
evaluated populations.

| | | Evaluated | |
|---|---|---|---|
| | | by PD-L1 | Evaluated |
| | Overall | expression | by PD-L1 |
| | population | on tumor | CPS |
| Characteristic, n (%) | N = 163 | n = 130 | n = 104 |
| Treatment arm | | | |
| NIVO3 | 59 (36) | 42 (32) | 32 (31) |
| NIVO3 + IPI1 | 52 (32) | 43 (33) | 36 (35) |
| NIVO1 + IPI3 | 49 (30) | 42 (32) | 33 (32) |
| NIVO1 + IPI1^{a} | 3 (2) | 3 (2) | 3 (3) |
| Age | | | |
| <65 | 117 (72) | 95 (73) | 78 (75) |
| ≥65 | 46 (28) | 35 (27) | 26 (25) |
| Sex | | | |
| Female | 37 (23) | 30 (23) | 26 (25) |
| Male | 126 (77) | 100 (77) | 78 (75) |
| ECOG performance status | | | |
| 0 | 75 (46) | 56 (43) | 45 (43) |
| 1 | 88 (54) | 74 (57) | 59 (57) |
| Disease site | | | |
| Esophagus | 26 (16) | 21 (16) | 14 (14) |
| Gastric | 61 (37) | 51 (39) | 43 (41) |
| Gastroesophageal junction | 76 (47) | 58 (45) | 47 (45) |

^{a}Three patients in the dose-escalation phase of NIVO1 + IPI1 were also included in the analysis.
CR, complete response; ECOG, Eastern Cooperative Oncology Group; NE, not evaluable; PD, progressive disease; PR, partial response; SD, stable disease.

PD-L1 immunohistochemistry (IHC) was used to evaluate PD-L1 expression on tumor and tumor-associated immune cells. Tumor PD-L1 expression, as used in the present example, represents the percentage of viable tumor cells showing partial or complete membrane PD-L1 staining. Tumor PD-L1 expression is calculated according to formula II:

$$\begin{matrix}Tumor \\ PD - L 1\end{matrix} = \frac{\# PD - L 1 Staining Cells ( Tumor Cells )}{Total \# Viable Tumor Cells}$$

Combined positive score (CPS) incorporates both tumor and tumor-associated immune cell PD-L1 expression. CPS is calculated according to formula III:

$$CPS = \frac{\begin{matrix}\# PD - L 1 Staining Cells ( Tumor Cells , \\ Lymphocytes , Macrophages )\end{matrix}}{Total \# Viable Tumor Cells} \times 100$$

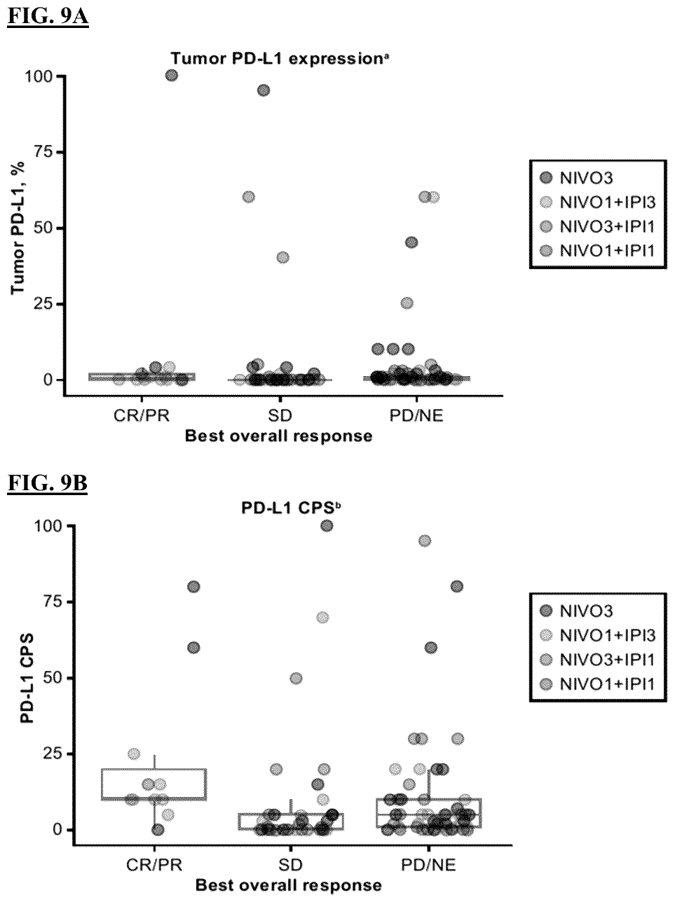

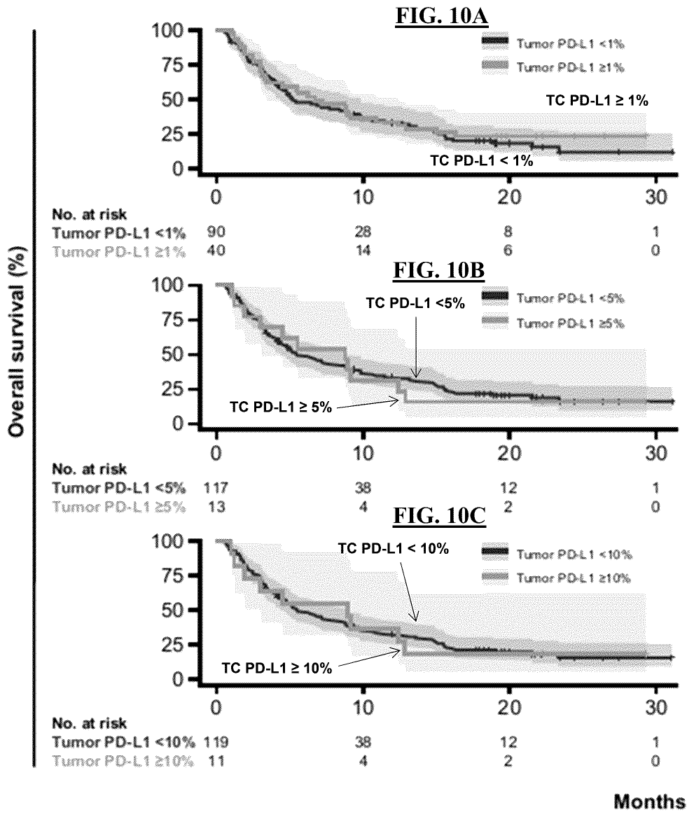

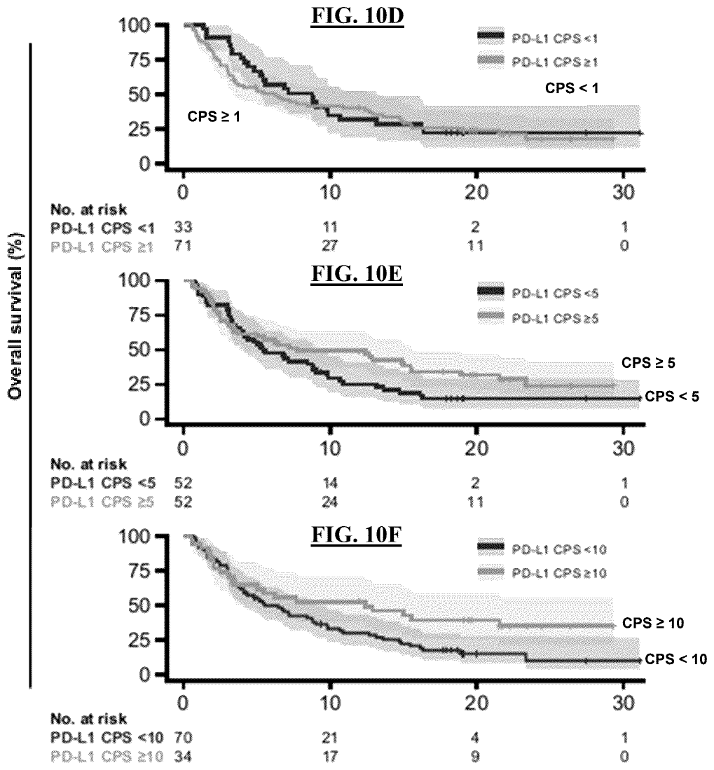

PD-L1 expression by CPS (FIG. 9B) was observed to have better association with response than PD-L1 expression on tumor cells (FIG. 9A). PD-L1 expression by CPS had a higher prevalence regardless of cutoff and had better association with response at higher cutoffs, as compared with PD-L1 expression on tumor cells (Table 5). At higher cutoffs, PD-L1 expression by CPS demonstrated a stronger association with overall survival than tumor PD-L1 expression (FIGS. 10A-10F).

TABLE 5
Prevalence and Response Rate by PD-L1 Expression on Tumor
Cells and by CPS: All Regimens

| | Prevalence, n (%) | | ORR, n (%) | |
|---|---|---|---|---|
| PD-L1 | Tumor PD-L1^{a} | PD-L1 CPS^{b} | Tumor PD-L1^{a} | PD-L1 CPS^{b} |
| cutoff^{c} | n = 130 | n = 104 | n = 130 | n = 104 |
| <1 | 90 (69) | 33 (32) | 7 (8) | 1 (3) |
| ≥1 | 40 (31) | 71 (68) | 7 (18) | 10 (14) |
| ≥5 | 13 (10) | 52 (50) | 1 (8) | 10 (19) |
| ≥10 | 11 (8) | 34 (33) | 1 (9) | 9 (27) |

^{a}PD-L1 expression on tumor cells
^{b}PD-L1 expression by CPS;
^{c}For tumor PD-L1 expression, the cutoff is represented as a percentage.
For CPS, the cutoff is represented as a score.
NA, not applicable;
ORR, objective response rate.

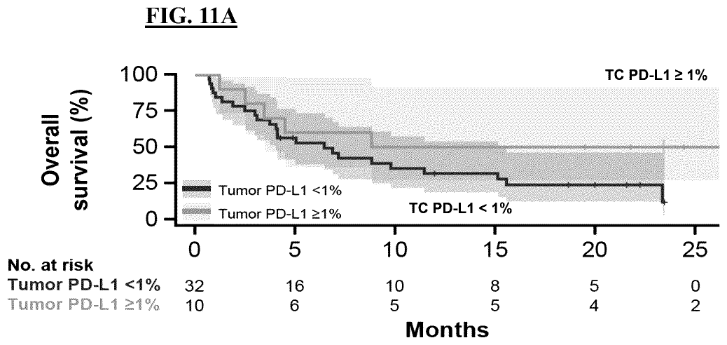

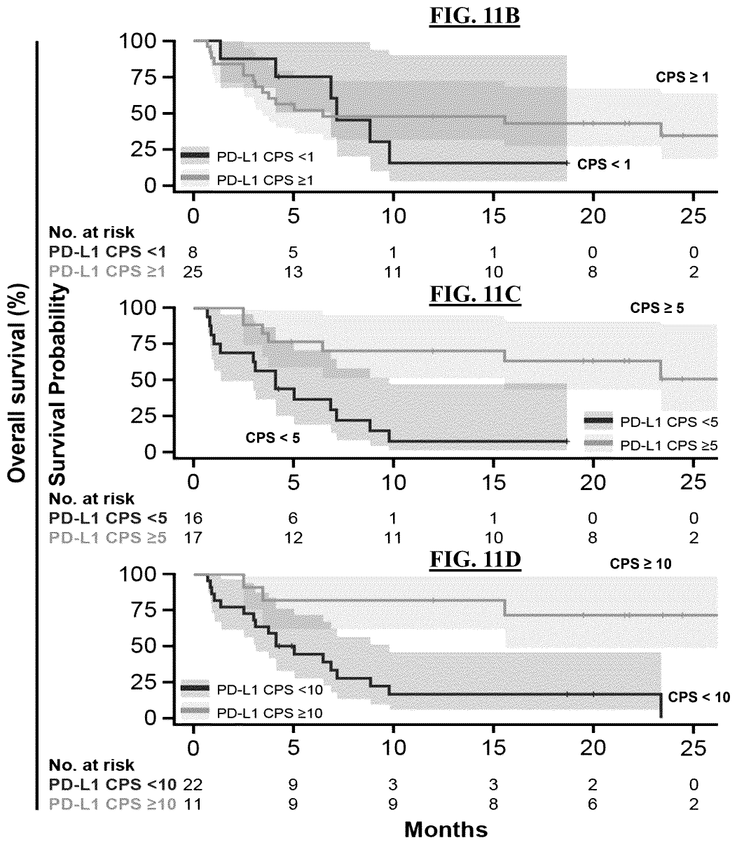

In the nivolumab 1 mg/kg+ipilimumab 3 mg/kg treatment arm, PD-L1 expression by CPS had a higher prevalence regardless of cutoff and had better association with response at higher cutoffs compared with PD-L1 expression on tumor cells. Further, PD-L1 expression by CPS demonstrated a stronger association with overall survival at higher cutoffs (FIGS. 11A-11D). This association in patients treated with nivolumab 1 mg/kg+ipilimumab 3 mg/kg was consistent with and more pronounced than in patients in all regimens combined (see FIGS. 10D-10F).

TABLE 6
Prevalence and Response Rate by PD-L1 Expression on Tumor
Cells and by CPS: nivolumab 1 mg/kg + ipilimumab 3 mg/kg.

| | Prevalence, n (%) | | Response rate, n (%) | |
|---|---|---|---|---|
| PD-L1 | Tumor PD-L1^{a} | PD-L1 CPS^{b} | Tumor PD-L1^{a} | PD-L1 CPS^{b} |
| cutoff^{c} | n = 42 | n = 33 | n = 42 | n = 33 |
| <1 | 32 (76) | 8 (24) | 6 (19) | 0 (0) |
| ≥1 | 10 (24) | 25 (76) | 4 (40) | 7 (28) |
| ≥5 | 1 (2^{d}) | 17 (52) | 0 (0) | 7 (41) |
| ≥10 | 1 (2^{d}) | 11 (33) | 0 (0) | 6 (55) |

^{a}PD-L1 expression on tumor cells
^{b}PD-L1 expression by CPS;
^{c}For tumor PD-L1 expression, the cutoff is represented as a percentage.
For CPS, the cutoff is represented as a score;
^{d}Only 1 patient had tumor PD-L1 ≥5% and ≥10%.

### Gene Profiling Analysis

Biological samples were collected from subjects prior to immunotherapy, and a subset of subject samples were available for gene expression profiling analysis (Table 7).

TABLE 7
Baseline characteristics and response: overall and
gene expression profile analysis populations.

| | Overall | GEP |
|---|---|---|
| | population | analysis |
| Characteristic, n (%) | N = 163 | n = 40 |
| Treatment arm | | |
| NIVO3 | 59 (33) | 11 (28) |
| NIVO3 + IPI1 | 52 (32) | 13 (33) |
| NIVO1 + IPI1 | 3 (2) | 2 (5) |
| NIVO1 + IPI3 | 49 (30) | 14 (35) |
| Age | | |
| <65 | 117 (72) | 30 (75) |
| ≥65 | 46 (28) | 10 (25) |
| Sex | | |
| Female | 37 (23) | 10 (25) |
| Male | 126 (77) | 30 (75) |
| ECOG performance status | | |
| 0 | 75 (46) | 12 (30) |
| 1 | 88 (54) | 28 (70) |
| Disease site | | |
| Esophagus | 26 (16) | 5 (13) |
| Gastric | 61 (37) | 13 (33) |
| Gastroesophageal junction | 76 (47) | 22 (55) |
| Response, n (%) | | |
| CR/PR | 16 (10) | 4 (10) |
| SD/PD/NE | 147 (90) | 36 (90) |

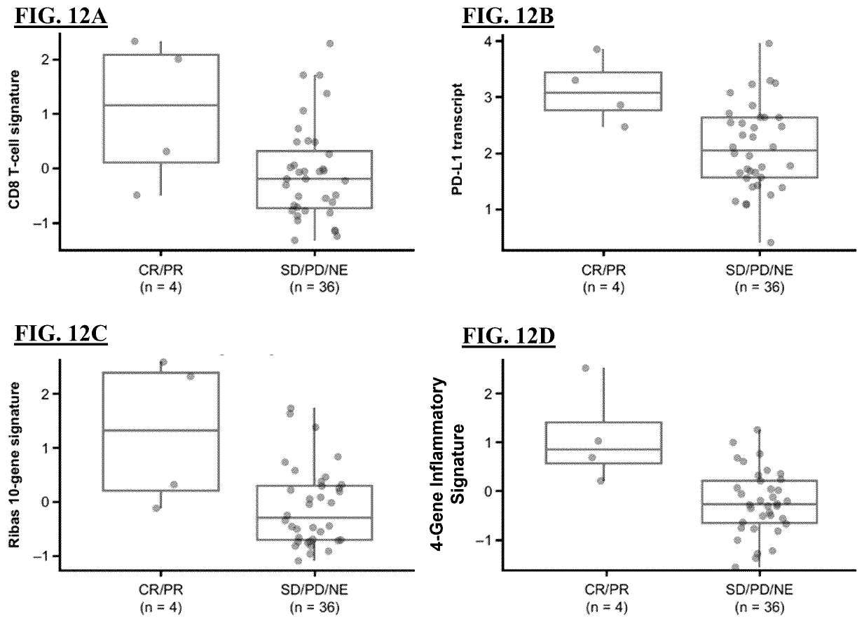

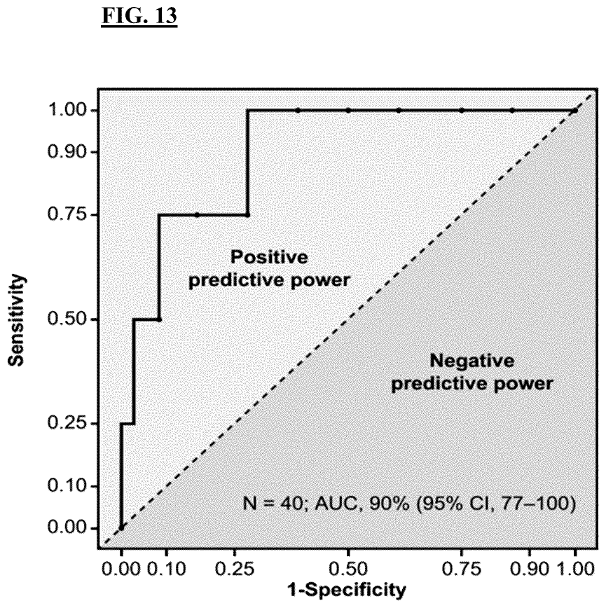

Various gene expression signatures were analyzed on available samples (Table 8). All gene expression signatures showed a trend in association with response (Table 8). Notably, significant associations were observed between the 4-gene inflammatory signature of the present disclosure (comprising CD274 (PD-L1), CD8A, LAG3, and STAT1; FIG. 12D), CD8 T-cell Signature (FIG. 12A), PD-L1 transcript (FIG. 12B), and the Ribas 10-Gene Interferon Gamma Signature (FIG. 12C), with the 4-gene inflammatory signature showing the strongest association with response (pateints with CR/PR, n=4; Table 8). Despite the small number of responding patients (n=4) for this analysis, good discrimination with AUC (90% [95% CI, 77-100]) was demonstrated (FIG. 13).

TABLE 8
Gene expression signatures and response.

| | | False |
|---|---|---|
| | | discovery |
| Gene signatures/transcripts | P-value^{a} | rate^{a,b} |
| 4-Gene Inflammatory Signature | 0.00411 | 0.037 |
| CD8 T-cell Signature¹ | 0.0321 | 0.0862 |
| Gajewski 13-Gene Inflammatory | 0.127 | 0.164 |
| Signature² | | |
| Interferon Gamma transcript | 0.0479 | 0.0862 |
| Ribas 10-Gene Interferon | 0.0416 | 0.0862 |
| Gamma Signature³ | | |
| PD-L1 transcript | 0.0621 | 0.0931 |
| T-cell Signature¹ | 0.171 | 0.18 |

^{a}P-value and false discovery rate derived from testing using 9 prespecified signatures and genes. False discovery rate adjusted P-value.
^{b}Estimate of the false discovery rate for a given number of tests/hypotheses.
^{c}Given the small sample size, exploratory P-values are intended to describe the relative performance of the different signatures for association with response.
¹Siemers NO, et al. PLoS One. 2017; 12: e0179726;
²Spranger S, et al. Nature. 2015; 523: 231-235;
³Ayers M, et al. J Clin Invest. 2017; 127: 2930-2940.

In this exploratory analysis, inflammatory gene signature expression was observed to be associated with response to nivolumab monotherapy and combination therapy with ipilimumab. This association indicates the presence of actionable biological factors that can be targeted by immuno-oncology agents

### Example 3: Genomic Analyses and Immunotherapy in Advanced Melanoma

Nivolumab (NIVO) and ipilimumab (IPI) are immune checkpoint inhibitors with distinct but complementary activity. Combination therapy comprising nivolumab and ipilimumab as well as nivolumab and ipilimumab monotherapies are approved for the treatment of unresectable or metastatic melanoma.

In studies of multiple tumors including melanoma, response to anti-PD-1 therapy was shown to associate with a T-cell inflamed gene expression profile.

This example reports the results of an exploratory analysis of an association of a novel inflammatory gene signature with clinical outcomes to nivolumab/ipilimumab combination therapy and nivolumab and ipilimumab monotherapies in melanoma.

### Study Design

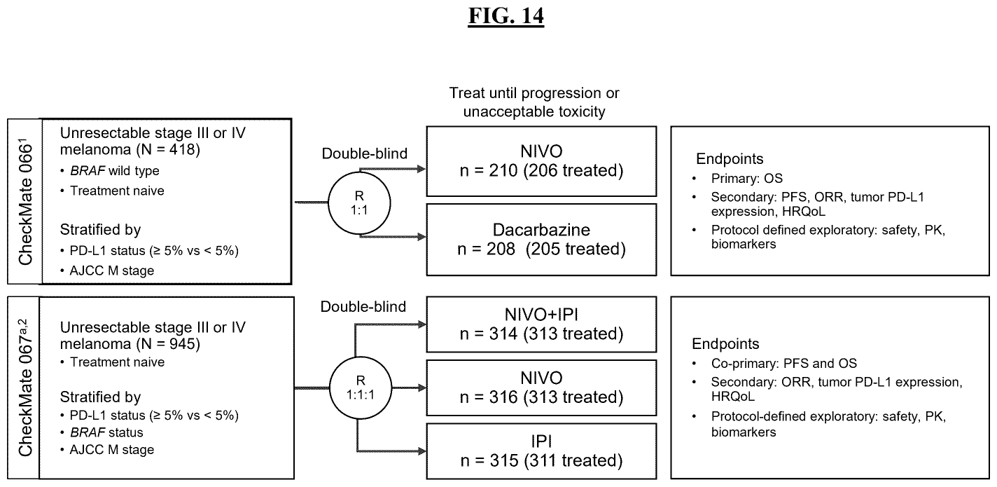

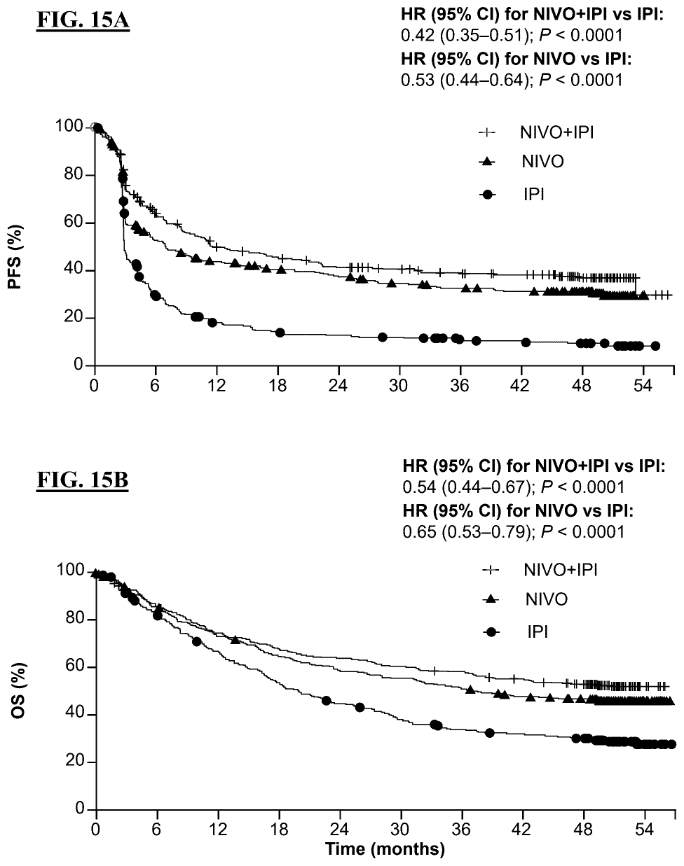

The present example reports data collected from clinical trials NCT01844505. In this trial, 945 previously untreated patients with unresectable stage III or IV melanoma were randomly assigned in a 1:1:1 ratio to receive one of the following regimens: (i) 3 mg of nivolumab per kilogram of body weight every 2 weeks (plus ipilimumab-matched placebo) (n=316, 313 treated); (ii) 1 mg of nivolumab per kilogram every 3 weeks plus 3 mg of ipilimumab per kilogram every 3 weeks for 4 doses, followed by 3 mg of nivolumab per kilogram every 2 weeks for cycle 3 and beyond (n=314, 313 treated); or 3 mg of ipilimumab per kilogram every 3 weeks for 4 doses (plus nivolumab-matched placebo) (n=315, 311 treated) (FIG. 14). Both nivolumab and ipilimumab were administered by means of intravenous infusion.

Randomization was stratified according to tumor PD-L1 status (positive vs. negative or indeterminate), BRAF mutation status (V600 mutation-positive vs. wild-type), and American Joint Committee on Cancer metastasis stage (M0, M1a, or M1b vs. M1c). Treatment continued until disease progression (as defined by RECIST, version 1.1), development of unacceptable toxic events, or withdrawal of consent.

Progression-free survival and overall survival were coprimary endpoints. Secondary endpoints included objective response rate, tumor PD-L1 expression, and health related quality of life. Exploratory endpoints included safety, pharmacokinetics, and biomarker analysis.

The 4-year follow up of NCT01844505 demonstrated durable, sustained survival benefit with first-line nivolumab/ipilimumab combination therapy and nivolumab monotherapy in patients with advanced melanoma (ORRb, % (95% CI): 58% (52.6-63.8) NIVO+IPI; 45% (39.1-50.3) NIVO; 19% (14.9-23.8) IPI; median PFS, months (95% CI): 11.5 (8.7-19.3) NIVO+IPI; 6.9 (5.1-10.2) NIVO; 2.9 (2.8-3.2) IPI; and median OS, months (95% CI): NR (38.2-NR) NIVO+IPI; 36.9 (28.3-NR) NIVO; 19.9 (16.9-24.6) IPI) (FIGS. 15A-15B). NCT01844505 was not powered for formal statistical comparison between nivolumab/ipilimumab combination therapy and nivolumab monotherapy.

### Objectives

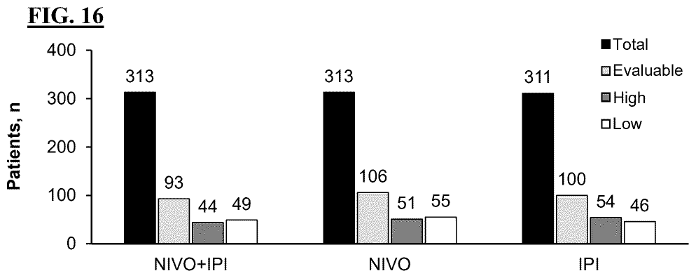

The objective of this analysis is to assess the association of inflammatory signature with clinical response, PFS, and OS with nivolumab-based immuno-oncology (I-O) therapy. For the inflammatory signature analysis, pretreatment tumor samples were analyzed using RNAseq to estimate relative tumor inflammation using the expression of 4 key genes—CD274 (PD-L1), CD8a, LAG3, and STAT1—comprising the 4-gene inflammatory signature, described herein. PFS and OS associations with the 4-gene inflammatory signature score were assessed in NCT01844505 samples using the relative median score to define high vs low 4-gene inflammatory signature score (median=−0.0434). A summary of the sample dispositions is provided in Table 9 and FIG. 16.

TABLE 9
Sample dispositions NCT01844505.

| NCT01844505 | NIVO + IPI | NIVO | IPI | Total |
|---|---|---|---|---|
| 4-gene inflammatory | 93/313 | 106/313 | 100/311 | 299/937 |
| signature evaluable^{c}, n | | | | |
| High score | 44 (47%) | 51 (48%) | 54 (54%) | 149 (50%) |
| Low score | 49 (53%) | 55 (52%) | 46 (46%) | 150 (50%) |

### Results

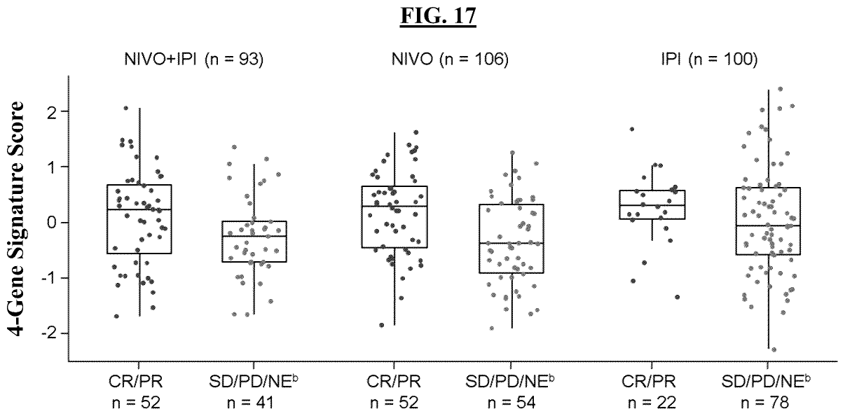

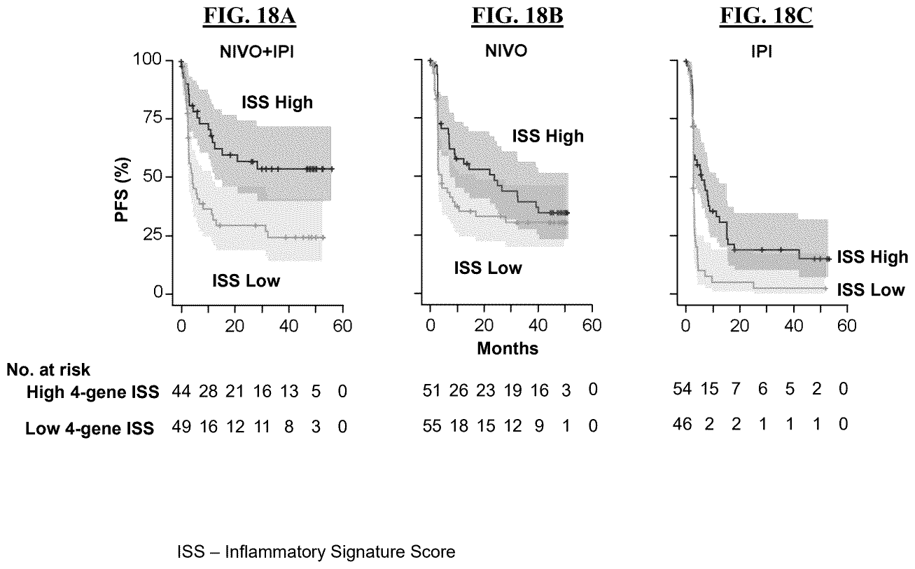

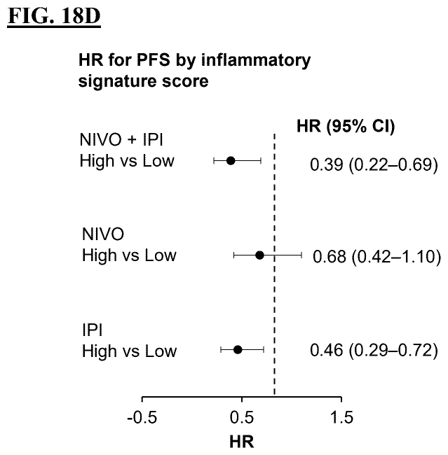

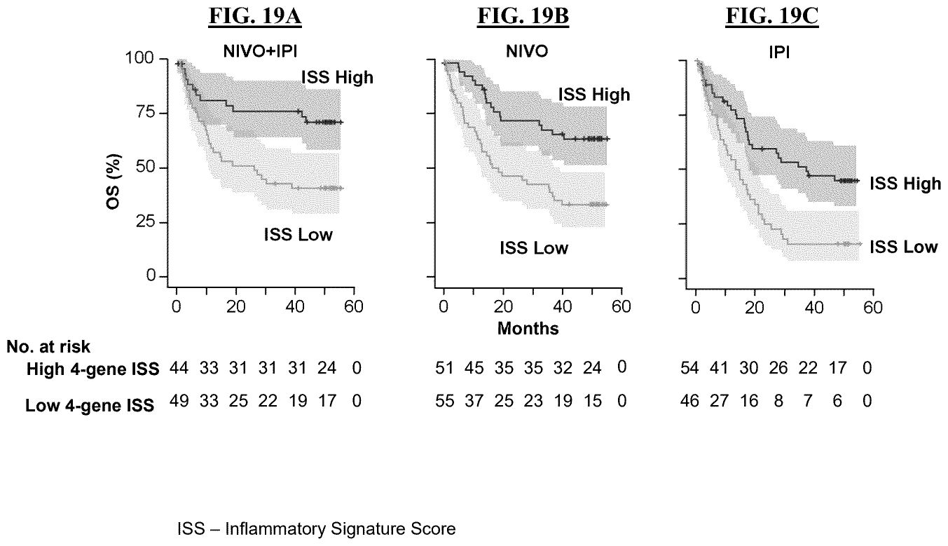

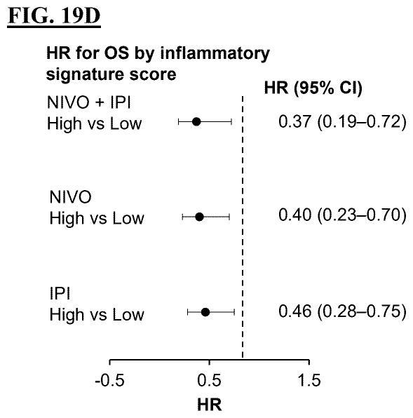

The distribution of 4-gene inflammatory signature score was higher in patients with response to treatment with nivolumab/ipilimumab combination therapy, nivolumab monotherapy, and ipilimumab monotherapy (FIG. 17). Longer PFS was observed for patients with high vs low inflammatory signature score across all treatment arms (FIGS. 18A-18D). Longer OS was also observed for patients with high vs low inflammatory signature score across all treatment arms (FIGS. 19A-19D).

In previously untreated metastatic melanoma, high 4-gene inflammatory signature score was observed to be associated with clinical response and increased survival with immuno-oncology therapy.

1. A method for treating a human subject afflicted with a tumor comprising administering to the subject an antibody or an antigen-binding portion that binds to the PD-1 receptor, wherein the subject is identified as exhibiting a high inflammatory signature score;
wherein the inflammatory signature score is determined by measuring the expression of inflammatory genes in an inflammatory gene panel in a tumor sample obtained from the subject, wherein the inflammatory genes consist of CD274 (PD-L1), CD8A, LAG3, and STAT1; and
wherein the tumor is derived from a cancer comprising hepatocellular cancer, gastroesophageal cancer, or melanoma.

2-8. (canceled)

9. The method of claim 1, wherein the high inflammatory signature score is characterized by an inflammatory signature score that is greater than an average inflammatory signature score, which is determined by averaging the expression of the panel of inflammatory genes in tumor samples obtained from a population of subjects afflicted with the tumor.

10. (canceled)

11. The method of claim 9, wherein the high inflammatory signature score is characterized by an inflammatory signature score that is at least about 25% higher than the average inflammatory signature score.

12-13. (canceled)

14. The method of claim 1, wherein the tumor sample comprises a tumor tissue biopsy.

15. The method of claim 1, wherein the tumor sample comprises a formalin-fixed, paraffin-embedded tumor tissue or a fresh-frozen tumor tissue.

16. The method of claim 1, wherein the measuring the expression of the inflammatory genes in the inflammatory gene panel comprises detecting the presence of inflammatory gene mRNA, the presence of a protein encoded by the inflammatory gene, or both.

17. The method of claim 16, wherein the presence of inflammatory gene mRNA is determined using reverse transcriptase PCR.

18. The method of claim 16, wherein the presence of the protein encoded by the inflammatory gene is determined using an IHC assay.

19-23. (canceled)

24. The method of claim 1, wherein the antibody or antigen-binding portion comprises nivolumab or an antigen-binding portion thereof.

25. The method of claim 1, wherein the antibody or antigen-binding portion comprises pembrolizumab or an antigen-binding portion thereof.

26. (canceled)

27. The method of claim 1, wherein the antibody or antigen-binding portion is administered at a dose of at least about 3 mg/kg body weight once about every 2 weeks.

28. (canceled)

29. The method of claim 1, wherein the antibody or antigen-binding portion thereof is administered at a flat dose of about 200, about 220, about 240, about 260, about 280, about 300, about 320, about 340, about 360, about 380, about 400, about 420, about 440, about 460, about 480, about 500 or about 550 mg.

30-32. (canceled)

33. The method of claim 1, wherein the antibody or antigen-binding portion thereof is administered at a flat dose of about 240 mg once about every two weeks.

34. The method of claim 1, wherein the antibody or antigen-binding portion thereof is administered at a flat dose of about 480 mg once about every four weeks.

35-37. (canceled)

38. The method of claim 1, further comprising administering an antibody or an antigen binding portion thereof that binds specifically to cytotoxic T-lymphocyte-associated protein 4 (CTLA-4) (“an anti-CTLA-4 antibody”).

39-40. (canceled)

41. The method of claim 38, wherein the anti-CTLA-4 antibody comprises ipilimumab or tremelimumab.

42-43. (canceled)

44. The method of claim 38, wherein the anti-CTLA-4 antibody is administered (i) at a dose of 1 mg/kg body weight once every 6 weeks, (ii) at a dose of 1 mg/kg body weight once every 4 weeks; or (iii) at a flat dose of about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg once about every 2, 3, 4, 5, 6, 7, or 8 weeks.

45-52. (canceled)

52. The method of claim 1, wherein the tumor is derived from a melanoma.

53. The method of claim 1, wherein the tumor is (i) relapsed, refractory, or both; (ii) locally advanced or metastatic; or (iii) both (i) and (ii).

54-66. (canceled)

67. A kit for treating a subject afflicted with a tumor, the kit comprising:
(a) a dosage ranging from about 4 mg to about 500 mg of an antibody or an antigen-binding portion that binds to the PD-1 receptor; and
(b) instructions for using the antibody or antigen-binding portion in the method of claim 1.

68-69. (canceled)
